(19) United States
(12) Patent Application Publication
(10) Pub. No.: US 2009/0042882 A1
(43) Pub. Date: Feb. 12, 2009

(54) SUBSTITUTED ACETAMIDES AS MODULATORS OF THE EP2 RECEPTOR

(76) Inventors: Bernd BUCHMANN, Hohen Neuendorf (DE); Nico Braeuer, Jena (DE); Olaf Peters, Tabarz (DE); Knut Eis, Berlin (DE); Marcus Koppitz, Berlin (DE); Bernhard Lindenthal, Berlin (DE); Gernot Langer, Falkensee (DE); Tim Wintermantel, Berlin (DE); Antonius Ter Laak, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/138,067

(22) Filed: Jun. 12, 2008

Related U.S. Application Data
(60) Provisional application No. 60/943,649, filed on Jun. 13, 2007.

(30) Foreign Application Priority Data
Jun. 13, 2007 (EP) 07090119.4

(51) Int. Cl. A61K 31/5377; C07D 209/20; C07D 413/12; A61P 15/08; A61P 35/00; A61K 31/404

(52) U.S. Cl. 514/235.2; 548/496; 514/419; 544/143

(57) ABSTRACT

The present invention relates to substituted acetamides of the general formula I, process for their preparation, and the use thereof for the manufacture of pharmaceutical compositions for the treatment of disorders and indications connected with the EP₂ receptor.

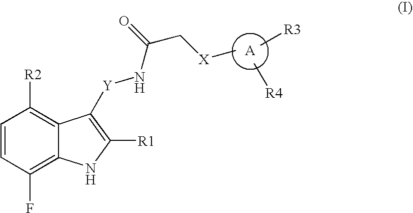

This application claims the benefit of the filing date of U.S. Provisional Application Ser. No. 60/943,649 filed Jun. 13, 2007.

The present invention relates to substituted acetamides as EP₂ receptor modulators, processes for their preparation, and their use as medicaments.

It has long been known that prostaglandins are key molecules in the processes of female reproductive biology such as, for example, control of ovulation, of fertilization, of nidation, of decidualization (e.g. placenta formation) and of menstruation. Prostaglandins likewise play an important part in the pathological changes in the reproductive tract, including menorrhagia, dysmenorrhea, endometriosis and cancer. The mechanism by which prostaglandins bring about these changes has not yet been completely elucidated. Recent results indicate that prostaglandins, their receptors and signal transduction pathways thereof are involved in processes such as angiogenesis, apoptosis, proliferation, and in inflammatory/antiinflammatory and immunological processes.

The effects of prostaglandins are mediated by their G protein-coupled receptors which are located on the cell surface. Prostaglandin E₂ (PGE₂) is of particular interest, having a wide variety of cellular effects through binding to functionally different receptor subtypes, namely the EP₁, EP₂, EP₃ and EP₄ receptors. The receptor subtypes to which prostaglandin E₂ binds appear to be of particular interest for the receptor-mediated effects which are involved in the control of fertility. It has thus been possible to show that the reproductive functions in EP₂ knockout mice (EP₂^{-/-}), i.e. in mice no longer having a functional PGE₂ receptor of the EP₂ subtype, are impaired, and that these animals have a smaller “litter size” (Matsumoto et al., 2001, Biology of Reproduction 64, 1557-1565). It was likewise possible to show that these EP₂ knockout mice (Hizaki et al. Proc Natl Acad Sci U.S.A. 1999 Aug. 31; 96 (18):10501-10506) show distinctly reduced cumulus expansion and severe subfertility, which is to be regarded as causally connected with diminished reproductive processes such as ovulation and fertilization.

The EP₂ receptor accordingly represents an important target for developing medicaments for controlling female fertility. The existence of the 4 subclasses of the PGE₂ receptor opens up the possibility of targeted development of selectively active compounds. However, to date, scarcely any selective EP₂ receptor ligands which bind to the EP₂ subtypes of the PGE₂ receptor are known, since most known compounds also bind to the other PGE₂ receptor subtypes such as, for example, to the EP₄ receptor.

EP₂ receptor antagonists are described, for example in the application US2005059742 (Jabbour, Medical Research Council). A method in which an EP₂ and/or an EP₄ antagonist can be employed for the treatment of menorrhagia and dysmenorrhea is claimed. AH6809 is disclosed as antagonist of the EP₂ or EP₄ receptor, but no other specific antagonists and no new compounds are disclosed.

In an earlier application of the same group (EP1467738), EP₂ or EP₄ antagonists are claimed for the treatment of pathological conditions such as, for example, allergic disorders, Alzheimer's disease, pain, abortion, painful menstruation, menorrhagia and dysmenorrhea, endometriosis, bone disorders, ischemia etc. The described compounds are, however, distinguished by a particularly high affinity for the EP₃ receptor. A further application (WO04/032964) describes novel compounds which are likewise distinguished by a particularly high affinity for the EP₃ receptor, but also have EP₂-antagonistic effects and which are used for the treatment and prophylaxis of allergic disorders.

Ono Pharmaceutical claims in the application WO03/016254 the preparation of benzene or saturated carboxylic acid derivatives which are substituted by aryl or heterocycles, inter alia as PGE₂ receptor antagonists. The disclosed compounds are claimed for the treatment of a large number of disorders, including allergic disorders, Alzheimer's disease, pain, abortion, painful menstruation, menorrhagia and dysmenorrhea, endometriosis, bone disorders, ischemia etc. The described compounds are, however, distinguished by a particularly high affinity for the EP₃ receptor. A further application (WO04/032964) describes novel compounds which are likewise distinguished by a particularly high affinity for the EP₃ receptor, but also have EP₂-antagonistic effects and which are used for the treatment and prophylaxis of allergic disorders.

The application WO04/39807 of Merck Frosst, Canada, discloses the preparation of pyridopyrrolizines and pyridoindolizines. However, these compounds are distinguished by good binding to the PGD₂ receptor, and this receptor represents a different subtype of the prostaglandin receptor.

Naphthalene derivatives as EP₄ receptor ligands are disclosed in application US2004102508 of SmithKline Beecham Corporation. The claimed compounds are used for the treatment or prophylaxis of pain, allergic reactions and neurodegenerative disorders.

EP₄ antagonists (γ-lactams) are claimed in the application WO03/103604 (Applied Research Systems). The compounds bind approximately 60-fold better to the EP₄ than to the EP₂ receptor and are claimed inter alia for the treatment of premature labor, dysmenorrhea, asthma, infertility or fertility impairments. The same company claims in the applications WO03/053923 (substituted pyrrolidines) or WO03/035064 (substituted pyrazolidinones) compounds for the treatment of disorders associated with prostaglandins, such as, for example, infertility, hypertension and osteoporosis. The compounds bind to the EP₄- and to the EP₂ receptor subtypes. The application WO03/037433 claims ω-cycloalkyl, 17 heteroaryl prostaglandin derivatives as EP₂ receptor antagonists, in particular for the treatment of elevated intraocular pressure.

The application WO03/064391 (Pfizer Products) describes metabolites of [3-[[N-(4-tert-butylbenzyl)(pyridin-3-ylsulfonyl)amino]methyl]acetic acid which inhibit the binding of [³H] prostaglandin E₂ to the EP₂ receptor. The use of these metabolites for the treatment of osteoporosis is disclosed.

Tani et al. claim in the application US2005124577 8-azaprostaglandin derivatives for the treatment of immunological disorders, allergic disorders, premature labor, abortion, etc. The compounds bind to the EP₂ and to the EP₄ receptor.

European patent application EP 1306087 describes EP₂ receptor agonists which are used for the treatment of erectile dysfunction (Ono Pharmaceuticals). The same class of structures is described in European patent EP 860430 (Ono Pharmaceuticals), and their use for the manufacture of a medicament for the treatment of immunological disorders, asthma and abortion is claimed. WO04/009117 describes EP₂ and EP₄ receptor agonists for the treatment of disorders caused by uterine contraction, for example painful menstruation (Ono Pharmaceuticals).

The applications WO03/74483 and WO03/09872 describe agonists which bind equally to the EP₂ and to the EP₄ receptor (Ono Pharmaceuticals).

Agonists of the EP₂ and of the EP₄ receptors are frequently described in connection with the treatment of osteoporosis (WO99/19300 (Pfizer), US2003/0166631 (Dumont Francis), WO03/77910 (Pfizer), WO03/45371 (Pfizer), WO03/74483 and WO03/09872 (Ono Pharmaceuticals)) and for glaucoma treatment (WO04/37813, WO04/37786, WO04/19938, WO03/103772, WO03/103664, WO03/40123, WO03/47513, WO03/47417 (Merck Frosst Canada)) and U.S. Pat. No. 6,410,591 and U.S. Pat. No. 6,747,037 (Allergan).

The patent application WO04/12656 (Applied Research Systems) claims EP₂ receptor agonists in connection with inflammation.

The patent application WO03/77919 (Merck & Co. Inc.) claims EP₄ receptor agonists for the treatment of fertility.

However, to date, no selective EP₂ receptor agonists and antagonists which control the processes which are ultimately responsible for ovulation, fertilization, nidation and decidualization and thus contribute to promoting or inhibiting fertility are known.

It is therefore an object of the present invention to provide stable EP₂ receptor antagonists.

This object is achieved by the compounds of the general formula I

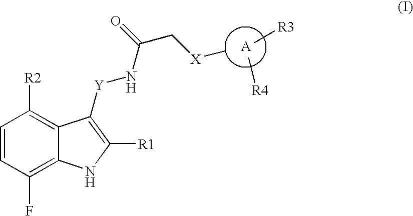

where
A is a C₆-C₁₂ aryl or C₅-C₁₆ heteroaryl radical which may optionally be substituted one or more times by R⁴ and/or R³,
R¹ is a hydrogen, a C₁-C₆-alkyl radical which may optionally be substituted,
R² is a hydrogen, halogen, cyano, —S(O)_{q}—CH₃, where q is 0-2, a C₁-C₄-alkoxy radical or C₁-C₆-alkyl radical, where this radical may be substituted in any way,
R³ is a hydrogen, halogen, amino, an —S(O)ₚ—C₁-C₆-alkyl group, where p is 0-2,
   an SO₂NH₂, COOH, CO—NH₂, NH—CO—NH₂, C₁-C₆-acyl-, —O—CO—NH—(C₁-C₆-alkyl), —O—CO—N—(C₁-C₆-alkyl)₂ or NH—CO—C₁-C₆-alkyl radical,
   a C₁-C₆-alkyl which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂,
   a C₅-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, CO—NH(C₁-C₆-alkyl), CO—N(C₁-C₆-alkyl)₂ or C₁-C₆-alkoxy,
R⁴ is a hydrogen, halogen, amino, —S(O)ₚ—C₁-C₆-alkyl, where p is 0-2,
   an SO₂NH₂, COOH, CO—NH₂, NH—CO—NH₂, C₁-C₆-acyl, NH—CO—C₁-C₆-alkyl, —O—CO—NH(C₁-C₆-alky), —O—CO—N(C₁-C₆-alkyl)₂,
   a C₁-C₆-alkyl which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂, a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂,
   a C₅-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, CO—NH(C₁-C₆-alkyl), CO—N(C₁-C₆-alkyl)₂ or C₁-C₆-alkoxy,
R³ and R⁴ are either in ortho position, meta position or meta, para position relative to one another and together have the meaning —O—CO—S—, —S—CO—O—, CH₂—CO—O—, O—CO—CH₂—,
   —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen, sulfur or an —NH group, and
Y is a —(CH₂)ₙ—, where n is 1-3,

and the isomers, diastereomers, enantiomers and salts thereof, and cyclodextrin clathrates, for the manufacture of a medicament for fertility control, which overcome the known disadvantages and have improved properties, i.e. a good activity, good solubility and stability.

The compounds of the invention have an antagonistic effect on the EP₂ receptor and thus serve to control female fertility.

C₁-C₆-alkyl means in each case a straight-chain or branched alkyl radical such as, for example, methyl, ethyl, propyl, isopropyl, butyl, isobutyl, sec-butyl, tert-butyl, pentyl, isopentyl and hexyl which may be substituted as desired one or more times, identically or differently, for example by halogen.

C₁-C₆-alkoxy or C₁-C₄-alkoxy means in each case a straight-chain or branched alkoxy radical such as, for example, methoxy-, ethoxy-, n-propoxy-, isopropoxy-, n-butoxy-, sec-butoxy-, isobutoxy-, tert-butyloxy-, pentoxy-, isopentoxy- and hexoxy which may optionally be substituted one or more times, identically or differently, for example by halogen.

C₁-C₆-acyl means in each case a straight-chain or branched radical such as, for example, formyl, acetyl, propionyl, butyryl, isobutyryl, valeryl and benzoyl which may optionally be substituted one or more times, identically or differently, for example by halogen.

C₃-C₆-Cycloalkyl means monocyclic 3-6-membered alkyl rings such as cyclopropyl, cyclobutyl, cyclopentyl and cyclohexyl.

The C₃-C₆-cycloalkyl radicals may, instead of the carbon atoms, comprise one or more heteroatoms such as oxygen, sulfur and/or nitrogen. Preferred heterocycloalkyls are those having 3 to 6 ring atoms such as, for example aziridinyl, azetidinyl, pyrrolidinyl, piperidinyl, piperazinyl, morpholinyl. Ring systems in which optionally one or more possible double bonds may be contained in the ring are for example cycloalkenyls such as cyclopropenyl, cyclobutenyl, cyclopentenyl, cyclopentadienyl, cyclohexenyl, cycloheptenyl, with the connection possibly taking place either at the double bond or at the single bonds.

Halogen means in each case fluorine, chlorine, bromine or iodine.

The aryl radical includes in each case 6-12 carbon atoms and may for example be benzo-fused. Examples which may be mentioned are: phenyl, tropyl, cyclooctadienyl, indenyl, naphthyl, biphenyl, fluorenyl, anthracenyl etc.

The heteroaryl radical comprises in each case 5-16 ring atoms and may, instead of the carbon, comprise one or more, identical or different, heteroatoms such as oxygen, sulfur or nitrogen in the ring, and may be mono-, bi- or tricyclic and may additionally in each case be benzo-fused.

Examples which may be mentioned are:

thienyl, furanyl, pyrrolyl, oxazolyl, imidazolyl, pyrazolyl, isoxazolyl, isothiazolyl, oxadiazolyl, triazolyl, thiadiazolyl, etc. and benzo derivatives thereof, such as, for example, benzofuranyl, benzothienyl, benzooxazolyl, benzimdazolyl, indazolyl, indolyl, isoindolyl, etc; or pyridazinyl, pyrimidinyl, pyrazinyl, triazinyl, etc. and benzo derivatives thereof such as, for example, quinolyl, isoquinolyl, etc; or azocinyl, indolizinyl, purinyl, etc. and benzo derivatives thereof; or quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl, oxepinyl, etc.

The heteroaryl radical may in each case be benzo-fused. Examples of 5-membered heteroaromatic rings which may be mentioned are: thiophene, furan, oxazole, thiazole, imidazole, pyrazole and benzo derivatives thereof, and of 6-membered heteroaromatic rings pyridine, pyrimidine, triazine, quinoline, isoquinoline and benzo derivatives.

Heteroatoms mean oxygen, nitrogen or sulfur atoms.

If an acidic function is present, suitable salts are the physiologically tolerated salts of organic and inorganic bases, such as, for example, the readily soluble alkali metal and alkaline earth metal salts, and N-methylglucamine, dimethylglucamine, ethylglucamine, lysine, 1,6-hexadiamine, ethanolamine, glucosamine, sarcosine, serinol, tris-hydroxymethylaminomethane, aminopropanediol, Sovak base, 1-amino-2,3,4-butanetriol.

If a basic function is present, the physiologically tolerated salts of organic and inorganic acids are suitable, such as hydrochloric acid, sulfuric acid, phosphoric acid, citric acid, tartaric acid inter alia.

Preference is given to those compounds of the general formula (I)

where
A is a C₆-C₁₂-aryl or C₅-C₁₆ heteroaryl radical which may optionally be substituted one or more times by R⁴ and/or R³,
R¹ is a hydrogen or C₁-C₆-alkyl radical which may be substituted one or more times by halogen,
R² is a hydrogen, halogen, cyano, an —S(O)_{q}—CH₃, where q is 0-2, a C₁-C₄-alkoxy radical or C₁-C₆-alkyl radical which may be substituted one or more times by halogen,
R³ is a hydrogen, halogen, amino, —S(O)ₚ—C₁-C₆-alkyl, where p is 0-2,
   an SO₂NH₂, COOH, CO—NH₂, NH—CO—NH₂, C₁-C₆-acyl-, NH—CO—C₁-C₆-alkyl, —O—CO—NH—(C₁-C₆-alkyl), —O—CO—N(C₁-C₆-alkyl)₂,
   a C₁-C₆-alkyl which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂,
   a C₅-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, CO—NH(C₁-C₆-alkyl), CO—N(C₁-C₆-alkyl)₂ or C₁-C₆-alkoxy,
R⁴ is a hydrogen, halogen, amino, —S(O)ₚ—C₁-C₆-alkyl, where p is 0-2,
   an SO₂NH₂, COOH, CO—NH₂, NH—CO—NH₂, C₁-C₆-acyl, NH—CO—C₁-C₆-alkyl, —O—CO—NH—(C₁-C₆-alkyl), —O—CO—N(C₁-C₆)-alkyl₂,
   a C₁-C₆-alkyl which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂,
   a C₅-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, CO—NH(C₁-C₆-alkyl), CO—N(C₁-C₆-alkyl)₂ or C₁-C₆-alkoxy,
R³ and R⁴ are either in ortho position, meta position or meta, para position relative to one another and together have the meaning —O—CO—S—, —S—CO—O—, CH₂—CO—O—, O—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen, sulfur or an —NH group, and
Y is a —(CH₂)ₙ—, where n is 1-3,

and the isomers, diastereomers, enantiomers and salts thereof, and cyclodextrin clathrates.

Particular preference is given to those compounds of the general formula (I)

where
A is a C₆-C₁₂-aryl or C₅-C₁₆ heteroaryl radical which may optionally be substituted one or more times by R⁴ and/or R³,
R¹ is a hydrogen or a C₁-C₆-alkyl radical which is substituted one or more times by halogen,
R² is a hydrogen, halogen, cyano, —S(O)_{q}—CH₃, where q is 0-2, a C₁-C₄-alkoxy radical or a C₁-C₆-alkyl group which is substituted one or more times by halogen,
R³ is a hydrogen, halogen, amino, —S(O)ₚ—CH₃, where p is 0-2,
   an —S—CF₃—, SO₂NH₂, COOH, CO—NH₂, NH—CO—NH₂, C₁-C₆-acyl, NH—CO—C₁-C₆-alkyl, —O—CO—NH—(C₁-C₆-alkyl), —O—CO—N(C₁-C₆-alkyl)₂,
   a C₁-C₆-alkyl which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
   a C₆-C₁₂-aryl which may optionally be substituted one or more times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
   a C₅-C₁₂-heteroaryl which may optionally be substituted one or more times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂, or
   a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R⁴ is a hydrogen, halogen, amino, —S(O)ₚ—C₁-C₆-alkyl, where p=0-2,
   an SO₂NH₂, COOH, CO—NH₂, NH—CO—NH₂, C₁-C₆-acyl-, NH—CO—C₁-C₆-alkyl, —O—CO—NH(C₁-C₆-alkyl), —O—CO—N(C₁-C₆-alkyl)₂,
   a C₁-C₆-alkyl which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
   a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂,
   a C₅-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂, or
   a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, CO—NH(C₁-C₆-alkyl), CO—N(C₁-C₆-alkyl)₂ or C₁-C₆-alkoxy,
R³ and R⁴ are either in ortho position, meta position or meta, para position relative to one another and together have the meaning —O—CO—S—, —S—CO—O—, CH₂—CO—O—, O—CO—CH₂—, O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen, sulfur or an —NH group, and
Y is a —(CH₂)ₙ—, where n is 1-3,

and the isomers, diastereomers, enantiomers and salts thereof, and cyclodextrin clathrates.

Preference is likewise given to those compounds of the general formula (I)

where
A is a C₆—C-aryl or C₅-C₁₆ heteroaryl radical which may optionally be substituted one or more times by R⁴ and/or R³,
R¹ is a hydrogen or a C₁-C₆-alkyl group which is substituted one or more times by halogen,
R² is hydrogen, halogen, cyano, —S(O)_{q}—CH₃, where q is 0-2, a C₁-C₄ alkoxy radical or C₁-C₆-alkyl group which is substituted one or more times by halogen,
R³ is a hydrogen, halogen, amino, —S(O)ₚ—CH₃, where p is 0-2,
   an —S—CF₃—, SO₂NH₂, COOH, CO—NH₂, NH—CO—NH₂, C₁-C₆-acyl-, NH—CO—C₁-C₆-alkyl, —O—CO—NHCH₃, —O—CO—N(CH₃)₂,
   a C₁-C₆-alkyl which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
   a C₆-C₁₂-aryl which may optionally be substituted one or more times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
   a C₅-C₁₂-heteroaryl which may optionally be substituted one or more times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂ or
   a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
   an —S—CF₃, SO₂NH₂, COOH, CO—NH₂, NH—CO—NH₂, C₁-C₆-acyl-, NH—CO—C₁-C₆-alkyl, —O—CO—NHCH₃, —O—CO—N(CH₃)₂,
   a C₁-C₆-alkyl which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
   a C₆-C₁₂-aryl which may optionally be substituted one or more times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
   a C₅-C₁₂-heteroaryl which may optionally be substituted one or more times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂, or
   a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R³ and R⁴ are either in ortho position, meta position or meta, para position relative to one another and together have the meaning —O—CO—S—, —S—CO—O—, CH₂—CO—O—, O—CO—CH₂—, —O—CO—NH—, —NH—CO—O—, —CO —CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen, sulfur or an —NH group, and
Y is a —(CH₂)ₙ, where n is 1-3,

and the isomers, diastereomers, enantiomers and salts thereof, and cyclodextrin clathrates.

Preference is likewise given to those compounds of the general formula (I)

where
A is a phenyl, naphthyl or C₅-C₁₆ heteroaryl radical which may be substituted by R³ and/or R⁴,
R¹ is a hydrogen or a C₁-C₆-alkyl group which may be substituted one or more times by halogen,
R² is a hydrogen, halogen, cyano, —S(O)_{q}—CH₃, where q is 0-2, a C₁-C₄-alkoxy radical or C₁-C₆-alkyl group,
R³ is a hydrogen, halogen, amino, —S(O)ₚ—CH₃, where p is 0-2,
   an —S—CF₃—, SO₂NH₂, COOH, CO—NH₂, NH—CO—NH₂, C₁-C₆-acyl, NH—CO—C₁-C₆-alkyl, —O—CO—NHCH₃, —O—CO—N(CH₃)₂,
   a C₁-C₆-alkyl which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
   a C₆-C₁₂-aryl which may optionally be substituted once, twice, three, four or five times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
   a C₅-C₁₂-heteroaryl which may optionally be substituted once, twice, three, four or five times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂ or
   a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2, an —S—CF₃, SO₂NH₂, COOH, CO—NH₂, NH—CO—NH₂, C₁-C₆-acyl-, NH—CO—C₁-C₆-alkyl, —O—CO—NHCH₃, —O—CO—N(CH₃)₂,
   a C₁-C₆-alkyl which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
   a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
   a C₆-C₁₂-aryl which may optionally be substituted once, twice, three, four or five times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
   a C₅-C₁₂-heteroaryl which may optionally be substituted once, twice, three, four or five times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂, or
   a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R³ and R⁴ are either in ortho position, meta position or meta, para position relative to one another and together have the meaning —O—CO—S—, —S—CO—O—, CH₂—CO—O—, O—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂(CH₂)ₘ—CH₂—, where m is 1-3,
X is an oxygen, sulfur or an —NH group, and
Y is a —(CH₂)ₙ—, where n is 1-3,

and the isomers, diastereomers, enantiomers and salts thereof, and cyclodextrin clathrates.

Preference is likewise given to those compounds of the general formula (I) where
A is a phenyl, naphthyl or C₅-C₁₆ heteroaryl radical which is substituted by R³ and/or R⁴,
R¹ is a hydrogen or a C₁-C₆-alkyl radical which is substituted one or more times by halogen,
R² is a hydrogen, halogen, cyano, —S(O)_{q}—CH₃, where q is 0-2, a C₁-C₄-alkoxy radical or C₁-C₆-alkyl radical which is substituted one or more times by halogen,
R³ is a hydrogen, halogen, amino, —S(O)ₚ—CH₃, where p is 0-2,
   an —S—CF₃—, SO₂NH₂, COOH, CO—NH₂, NH—CO—NH₂, C₁-C₆-acyl, NH—CO—C₁-C₆-alkyl, —O—CO—NHCH₃, —O—CO—N(CH₃)₂,
   a C₁-C₆-alkyl which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
   a C₁-C₄-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₄-alkyl), O—CO—(C₁-C₄-alkyl), N—(C₁-C₄-alkyl)₂, NH—(C₁-C₄-alkyl), CO—NH(C₅-C₁₂-heteroaryl), CO—NH(C₁-C₄-alkyl), CO—N(C₁-C₄-alkyl)₂,
   a C₆-C₁₂-aryl which may optionally be substituted once, twice, three, four or five times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
   a C₅-C₁₂-heteroaryl which may optionally be substituted once, twice, three, four or five times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂, or
   a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃—, where p is 0-2,
   an —S—CF₃, SO₂NH₂—, COOH, CO—NH₂, NH—CO—NH₂, C₁-C₆-acyl-, NH—CO—C₁-C₆-alkyl, —O—CO—NHCH₃, —O—CO—N(CH₃)₂,
   a C₁-C₆-alkyl which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
   a C₁-C₄-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₄-alkyl), O—CO—(C₁-C₄-alkyl), N—(C₁-C₄-alkyl)₂, NH—(C₁-C₄-alkyl), CO—NH(C₅-C₁₂-heteroaryl), CO—NH(C₁-C₄-alkyl), CO—N(C₁-C₄-alkyl)₂,
   a C₆-C₁₂-aryl which may optionally be substituted once, twice, three, four or five times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH₂, CO—NH(C₁-C₄-alkyl) or CO—N(C₁-C₄-alkyl)₂,
   a C₅-C₁₂-heteroaryl which may optionally be substituted once, twice, three, four or five times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂, or
   a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R³ and R⁴ are either in ortho position, meta position or meta, para position relative to one another and together have the meaning —O—CO—S—, —S—CO—O—, CH₂—CO—O—, O—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen, sulfur or an —NH group, and
Y is a —(CH₂)ₙ—, where n is 1-3,

and the isomers, diastereomers, enantiomers and salts thereof, and cyclodextrin clathrates.

Preference is likewise given to those compounds of the general formula (I), where
A is a phenyl, naphthyl or C₅-C₁₆ heteroaryl radical which is substituted by R³ and/or R⁴,
R¹ is a hydrogen or a C₁-C₆-alkyl radical which is substituted one or more times by halogen,
R² is hydrogen, halogen, cyano, —S(O)_{q}—CH₃, where q is 0-2, a C₁-C₄-alkoxy radical or C₁-C₆-alkyl radical which is substituted one or more times by halogen,
R³ is a hydrogen, halogen, amino, —S(O)ₚ—CH₃, where p is 0-2,
   an —S—CF₃—, SO₂NH₂, COOH, CO—NH₂, NH—CO—NH₂, C₁-C₆-acyl, NH—CO—C₁-C₆-alkyl, —O—CO—NHCH₃, —O—CO—N(CH₃)₂,
   a C₁-C₆-alkyl which may optionally be substituted once or twice, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
   a C₁-C₄-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₄-alkyl), O—CO—(C₁-C₄-alkyl), N—(C₁-C₄-alkyl)₂, NH—(C₁-C₄-alkyl), CO—NH(C₅-C₁₂-heteroaryl), CO—NH(C₁-C₄-alkyl), CO—N(C₁-C₄-alkyl)₂,
   a C₆-C₁₂-aryl which may optionally be substituted once or twice, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
   a C₅-C₁₂-heteroaryl which may optionally be substituted once or twice, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂ or
   a C₃-C₆-cycloalkyl which may optionally be substituted once or twice, identically or differently, by halogen, by C₁-C₄-alkyl, hydroxy, cyano, CO₂—(C₁-C₄-alkyl), C₁-C₄-alkyl, N—(C₁-C₄-alkyl)₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2, an —S—CF₃, SO₂NH₂, COOH, CO—NH₂, NH—CO—NH₂, C₁-C₆-acyl-, NH—CO—C₁-C₆-alkyl, —O—CO—NHCH₃, —O—CO—N(CH₃)₂,
   a C₁-C₆-alkyl which may optionally be substituted once or twice, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
   a C₁-C₄-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
   an —O—C₆-C₁₂-aryl, —CH₂—O—C₆-C₁₂-aryl, —O—C₅-C₁₆-heteroaryl, hydroxy, cyano, CO₂—(C₁-C₄-alkyl), O—CO—(C₁-C₄-alkyl), N—(C₁-C₄-alkyl)₂, NH—(C₁-C₄-alkyl), CO—NH(C₅-C₁₂-heteroaryl), CO—NH(C₁-C₄-alkyl), CO—N(C₁-C₄-alkyl)₂,
   a C₆-C₁₂-aryl which may optionally be substituted once or twice, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
   a C₅-C₁₂-heteroaryl which may optionally be substituted once or twice, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂, or
   a C₃-C₆-cycloalkyl which may optionally be substituted once or twice, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH—(C₁-C₄-alkyl), CO—N(C₁-C₄-alkyl)₂ or C₁-C₄-alkoxy,
R³ and R⁴ are either in ortho, meta position or meta, para position relative to one another and together have the meaning —O—CO—S—, —S—CO—O—, CH₂—CO—O—, O—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen or sulfur, and
Y is a —(CH₂)ₙ—, where n is 1-3,

and the isomers, diastereomers, enantiomers and salts thereof, and cyclodextrin clathrates.

The following compounds corresponding to the present invention are very particularly preferred:
1. N-[2-(7-fluoro-2,4-dimethyl-1H-indol-3-yl)ethyl]-2-(4-methoxyphenoxy)-acetamide
2. N-[2-(7-fluoro-2,4-dimethyl-1H-indol-3-yl)ethyl]-2-(3-methoxyphenoxy)-acetamide
3. N-[2-(7-fluoro-2,4-dimethyl-1H-indol-3-yl)ethyl]-2-(2-methoxyphenoxy)-acetamide
4. N-[2-(7-fluoro-2,4-dimethyl-1H-indol-3-yl)ethyl]-2-(4-fluorophenylthio)-acetamide
5. N-[2-(7-fluoro-2,4-dimethyl-1H-indol-3-yl)ethyl]-2-phenoxyacetamide
6. 2-(4-chlorophenoxy)-N-[2-(7-fluoro-2,4-dimethyl-1H-indol-3-yl)ethyl]-acetamide
7. 2-(2-chlorophenoxy)-N-[2-(7-fluoro-2,4-dimethyl-1H-indol-3-yl)ethyl]-acetamide
8. N-[2-(7-fluoro-2,4-dimethyl-1H-indol-3-yl)ethyl]-2-(isoquinolin-7-yloxy)-acetamide
9. 2-(3-acetylaminophenoxy)-N-[2-(7-fluoro-2,4-dimethyl-1H-indol-3-yl)ethyl]-acetamide
10. N-[2-(7-fluoro-2,4-dimethyl-1H-indol-3-yl)ethyl]-2-(5,6,7,8-tetrahydro-naphthalen-2-yloxy)acetamide
11. 4-{[2-(7-fluoro-2,4-dimethyl-1H-indol-3-yl)ethylcarbamoyl]methoxy}benzoic acid methylester
12. 2-(biphenyl-4-yloxy)-N-[2-(7-fluoro-2,4-dimethyl-1H-indol-3-yl)ethyl]-acetamide
13. 2-(4-acetylaminophenoxy)-N-[2-(7-fluoro-2,4-dimethyl-1H-indol-3-yl)ethyl]-acetamide
14. N-[2-(7-fluoro-2,4-dimethyl-1H-indol-3-yl)ethyl]-2-(2-oxo-benzo[1,3]oxathiol-6-yloxy)acetamide
15. 2-(3-cyanophenoxy)-N-[2-(7-fluoro-2,4-dimethyl-1H-indol-3-yl)ethyl]-acetamide
16. N-[2-(7-fluoro-2,4-dimethyl-1H-indol-3-yl)ethyl]-2-(quinolin-8-yloxy)-acetamide
17. 2-(5-chloro-2-methylphenoxy)-N-[2-(7-fluoro-2,4-dimethyl-1H-indol-3-yl)-ethyl]acetamide
18. N-[2-(7-fluoro-2,4-dimethyl-1H-indol-3-yl)ethyl]-2-(1-oxo-indan-5-yloxy)-acetamide
19. N-[2-(7-fluoro-2,4-dimethyl-1H-indol-3-yl)ethyl]-2-o-tolyloxyacetamide
20. 2-(4-chloro-2-methylphenoxy)-N-[2-(7-fluoro-2,4-dimethyl-1H-indol-3-yl)-ethyl]acetamide
21. N-[2-(7-fluoro-2,4-dimethyl-1H-indol-3-yl)ethyl]-2-m-tolyloxyacetamide
22. N-[2-(7-fluoro-2,4-dimethyl-1H-indol-3-yl)ethyl]-2-p-tolyloxyacetamide
23. N-[2-(7-fluoro-2,4-dimethyl-1H-indol-3-yl)ethyl]-2-(naphthalen-2-yloxy)-acetamide
24. N-[2-(7-fluoro-2,4-dimethyl-1H-indol-3-yl)ethyl]-2-(5,6,7,8-tetrahydro-naphthalen-1-yloxy)acetamide
25. N-[2-(7-fluoro-2,4-dimethyl-1H-indol-3-yl)ethyl]-2-(naphthalen-1-yloxy)-acetamide
26. 2-(4-chloro-3-methylphenoxy)-N-[2-(7-fluoro-2,4-dimethyl-1H-indol-3-yl)-ethyl]acetamide
27. N-[2-(7-fluoro-2,4-dimethyl-1H-indol-3-yl)ethyl]-2-(3-fluorophenoxy)-acetamide
28. 2-(3,5-dimethoxyphenoxy)-N-[2-(7-fluoro-2,4-dimethyl-1H-indol-3-yl)ethyl]-acetamide
29. N-[2-(7-fluoro-2,4-dimethyl-1H-indol-3-yl)ethyl]-2-(4-imidazol-1-ylphenoxy)-acetamide
30. 2-(3,4-dimethoxyphenoxy)-N-[2-(7-fluoro-2,4-dimethyl-1H-indol-3-yl)ethyl]-acetamide
31. 2-(biphenyl-2-yloxy)-N-[2-(7-fluoro-2,4-dimethyl-1H-indol-3-yl)ethyl]-acetamide
32. N-[2-(7-fluoro-2,4-dimethyl-1H-indol-3-yl)ethyl]-2-(1H-indol-4-yloxy)-acetamide
33. N-[2-(7-fluoro-2,4-dimethyl-1H-indol-3-yl)ethyl]-2-(1H-indol-5-yloxy)-acetamide
34. 2-(4-tert-butylphenoxy)-N-[2-(7-fluoro-2,4-dimethyl-1H-indol-3-yl)ethyl]-acetamide
35. 2-(4-{[2-(7-fluoro-2,4-dimethyl-1H-indol-3-yl)ethylcarbamoyl]methoxy}-phenyl)acetamide
36. 2-(benzo[1,3]dioxol-5-yloxy)-N-[2-(7-fluoro-2,4-dimethyl-1H-indol-3-yl)ethyl]-acetamide
37. N-[2-(7-fluoro-2,4-dimethyl-1H-indol-3-yl)ethyl]-2-(2-pyrrol-1-ylphenoxy)-acetamide
38. N-[2-(7-fluoro-2,4-dimethyl-1H-indol-3-yl)ethyl]-2-(4-isopropylphenoxy)-acetamide
39. N-[2-(7-fluor-2,4-dimethyl-1H-indol-3-yl)ethyl]-2-(quinazolin-4-yloxy)-acetamide
40. N-[2-(7-fluoro-2,4-dimethyl-1H-indol-3-yl)ethyl]-2-(6-methylpyridin-3-yloxy)-acetamide
41. N-[2-(7-fluoro-2,4-dimethyl-1H-indol-3-yl)ethyl]-2-(3-piperidin-4-ylphenoxy)-acetamide
42. N-[2-(7-fluoro-2,4-dimethyl-1H-indol-3-yl)ethyl]-2-(indan-5-yloxy)acetamide
43. N-[2-(7-fluoro-2,4-dimethyl-1H-indol-3-yl)ethyl]-2-(2-oxo-2,3-dihydro-benzofuran-5-yloxy)acetamide
44. 2-(3,4-difluorophenoxy)-N-[2-(7-fluoro-2,4-dimethyl-1H-indol-3-yl)ethyl]-acetamide
45. 2-(2-dimethylaminomethylphenoxy)-N-[2-(7-fluoro-2,4-dimethyl-1H-indol-3-yl)ethyl]acetamide
46. 2-(3-dimethylaminophenoxy)-N-[2-(7-fluoro-2,4-dimethyl-1H-indol-3-yl)ethyl]-acetamide
47. 2-(4-dimethylaminomethylphenoxy)-N-[2-(7-fluoro-2,4-dimethyl-1H-indol-3-yl)ethyl]acetamide
48. 2-(2,3-dimethoxyphenoxy)-N-[2-(7-fluoro-2,4-dimethyl-1H-indol-3-yl)ethyl]-acetamide
49. N-[2-(7-fluoro-2,4-dimethyl-1H-indol-3-yl)ethyl]-2-[2-(2H-pyrazol-3-yl)-phenoxy]acetamide
50. N-[2-(7-fluoro-2,4-dimethyl-1H-indol-3-yl)ethyl]-2-(4-pyrrol-1-ylphenoxy)-acetamide
51. N-[2-(7-fluoro-2,4-dimethyl-1H-indol-3-yl)ethyl]-2-(2-isoxazol-5-ylphenoxy)-acetamide
52. N-[2-(7-fluoro-2,4-dimethyl-1H-indol-3-yl)ethyl]-2-(2-methylbenzothiazol-5-yloxy)acetamide
53. 2-(2-chloro-4-fluorophenoxy)-N-[2-(7-fluoro-2,4-dimethyl-1H-indol-3-yl)ethyl]-acetamide
54. N-[2-(7-fluoro-2,4-dimethyl-1H-indol-3-yl)ethyl]-2-(3-morpholin-4-ylphenoxy)-acetamide
55. 2-(3,4-dimethylphenoxy)-N-[2-(7-fluoro-2,4-dimethyl-1H-indol-3-yl)ethyl]-acetamide
56. N-[2-(7-fluoro-2,4-dimethyl-1H-indol-3-yl)ethyl]-2-(4-methylpyridin-2-yloxy)-acetamide
57. N-[2-(7-fluoro-2,4-dimethyl-1H-indol-3-yl)ethyl]-2-(isoquinolin-5-yloxy)-acetamide
58. N-[2-(7-fluoro-2,4-dimethyl-1H-indol-3-yl)ethyl]-2-(4-phenoxyphenoxy)-acetamide
59. N-[2-(7-fluoro-2,4-dimethyl-1H-indol-3-yl)ethyl]-2-(quinolin-4-yloxy)acetamide
60. N-[2-(7-fluoro-2,4-dimethyl-1H-indol-3-yl)ethyl]-2-(4-oxazol-2-ylphenoxy)-acetamide
61. 2-(4-cyanophenoxy)-N-[2-(7-fluoro-2,4-dimethyl-1H-indol-3-yl)ethyl]-acetamide
62. 2-(2-chloro-5-methylphenoxy)-N-[2-(7-fluoro-2,4-dimethyl-1H-indol-3-yl)-ethyl]acetamide
63. 2-(biphenyl-3-yloxy)-N-[2-(7-fluoro-2,4-dimethyl-1H-indol-3-yl)ethyl]-acetamide
64. N-[2-(7-fluoro-2,4-dimethyl-1H-indol-3-yl)ethyl]-2-(4-fluorophenoxy)-acetamide
65. 2-(3-chlorophenoxy)-N-[2-(7-fluoro-2,4-dimethyl-1H-indol-3-yl)ethyl]-acetamide
66. 2-(3-chloro-4-fluorophenoxy)-N-[2-(7-fluoro-2,4-dimethyl-1H-indol-3-yl)ethyl]-acetamide
67. N-[2-(7-fluoro-2,4-dimethyl-1H-indol-3-yl)ethyl]-2-(3-methoxy-5-methyl-phenoxy)acetamide
68. N-[2-(7-fluoro-2,4-dimethyl-1H-indol-3-yl)ethyl]-2-(quinolin-7-yloxy)-acetamide
69. N-[2-(7-fluoro-2,4-dimethyl-1H-indol-3-yl)ethyl]-2-(quinolin-6-yloxy)-acetamide
70. N-[2-(7-fluoro-2,4-dimethyl-1H-indol-3-yl)ethyl]-2-(4-[1,2,4]triazol-1-yl-phenoxy)acetamide
71. N-[2-(7-fluoro-2,4-dimethyl-1H-indol-3-yl)ethyl]-2-(3-piperazin-1-ylphenoxy)-acetamide
72. N-[2-(7-fluoro-2,4-dimethyl-1H-indol-3-yl)ethyl]-2-(3-piperazin-1-ylphenoxy)-acetamide
73. N-[2-(7-fluoro-2,4-dimethyl-1H-indol-3-yl)ethyl]-2-(4-fluoro-2-methyl-1H-indol-5-yloxy)acetamide
74. N-[2-(7-fluoro-2,4-dimethyl-1H-indol-3-yl)ethyl]-2-(4-piperazin-1-ylphenoxy)-acetamide
75. 2-(3-chloro-2-methyl-phenoxy)-N-[2-(7-fluoro-2,4-dimethyl-1H-indol-3-yl)-ethyl]acetamide
76. 2-(2,3-difluorophenoxy)-N-[2-(7-fluoro-2,4-dimethyl-1H-indol-3-yl)ethyl]-acetamide
77. 2-(3-chloro-2-fluorophenoxy)-N-[2-(7-fluoro-2,4-dimethyl-1H-indol-3-yl)ethyl]-acetamide
78. N-[2-(7-fluoro-2,4-dimethyl-1H-indol-3-yl)ethyl]-2-(3-carbamoylamino-phenoxy)acetamide
79. 2-(9H-carbazol-3-yloxy)-N-[2-(7-fluoro-2,4-dimethyl-1H-indol-3-yl)ethyl]-acetamide
80. 2-(4-bromo-3-methoxyphenoxy)-N-[2-(7-fluoro-2,4-dimethyl-1H-indol-3-yl)-ethyl]acetamide
81. 2-(3-chloro-5-fluorophenoxy)-N-[2-(7-fluoro-2,4-dimethyl-1H-indol-3-yl)ethyl]-acetamide
82. 2-(2-chloro-4-methylphenoxy)-N-[2-(7-fluoro-2,4-dimethyl-1H-indol-3-yl)-ethyl]acetamide
83. 2-(3-chloro-4-methylphenoxy)-N-[2-(7-fluoro-2,4-dimethyl-1H-indol-3-yl)-ethyl]acetamide
84. N-[2-(7-fluoro-2,4-dimethyl-1H-indol-3-yl)ethyl]-2-(4-fluoro-3-methyl-phenoxy)acetamide
85. N-[2-(7-fluoro-2,4-dimethyl-1H-indol-3-yl)ethyl]-2-(3-fluoro-4-methyl-phenoxy)acetamide
86. N-[2-(7-fluoro-2,4-dimethyl-1H-indol-3-yl)ethyl]-2-(4-tetrazol-1-ylphenoxy)-acetamide
87. N-[2-(7-fluoro-2,4-dimethyl-1H-indol-3-yl)ethyl]-2-[(3-dimethylcarbamoyl)oxy]-phenoxy)acetamide
88. N-[2-(7-fluoro-2,4-dimethyl-1H-indol-3-yl)ethyl]-2-(4-fluoro-2-methyl-phenoxy)acetamide
89. N-[2-(7-fluoro-2,4-dimethyl-1H-indol-3-yl)ethyl]-2-(3-fluoro-5-methyl-phenoxy)acetamide
90. N-[2-(7-fluoro-2,4-dimethyl-1H-indol-3-yl)ethyl]-2-(2-oxo-2,3-dihydro-benzooxazol-6-yloxy)acetamide
91. N-[2-(7-fluoro-2,4-dimethyl-1H-indol-3-yl)ethyl]-2-[(4-dimethylcarbamoyl)oxy]-phenoxy)acetamide
92. 4-{[2-(7-fluoro-2,4-dimethyl-1H-indol-3-yl)ethylcarbamoyl]methoxy}-benzamide
93. 2-(9H-carbazol-2-yloxy)-N-[2-(7-fluoro-2,4-dimethyl-1H-indol-3-yl)ethyl]-acetamide

The present invention relates to the use of the compounds of the invention for manufacturing medicaments which comprise at least one of the compounds of formula I.

The present invention likewise relates to medicaments which comprise the compounds of the invention with suitable formulating substances and carriers.

Compared with known prostaglandin E₂ ligands, the novel EP₂ agonists and antagonists are distinguished by greater selectivity and stability.

The present invention relates to medicaments for the treatment and prophylaxis of disorders which include fertility impairments, infectious disorders, cancer, viral infections, cardiovascular disorders, elevated intraocular pressure, glaucoma, skeletal system disorders, angiogenetic disorders, uterine contraction impairments, pain, neuroinflammatory disorders, immunomodulatory infections and nephrological disorders.

Fertility impairments mean the disorders which lead to no ovulation taking place, that no nidation of a fertilized oocyte occurs and no decidualization takes place, infectious disorders mean disorders caused by unicellular parasites, cancer means solid tumors and leukemia, viral infections mean for example cytomegalus infections, hepatitis, hepatitis B and C and HIV disorders, immunomodulatory infections mean for example avian influenza, cardiovascular disorders mean ischemic reperfusion disorder, stenoses, arterioscleroses and restenoses, angiogenetic disorders mean for example endometriosis and fibrosis, elevated intraocular pressure means glaucoma, uterine contraction impairments mean for example painful menstruation, skeletal system disorders mean osteoporosis, neuroinflammatory disorders mean multiple sclerosis, Alzheimer's disease, pain and nephrological disorders mean glomerulonephritis.

The present invention likewise relates to medicaments for the treatment and prophylaxis of the disorders detailed above, which comprise at least one compound of the general formula I, and medicaments with suitable formulating substances and carriers.

For the compounds of the invention to be used as medicaments they are brought into the form of a pharmaceutical product which, besides the active ingredient, comprises inert organic or inorganic pharmaceutical carrier materials which are suitable for enteral or parenteral administration, such as, for example, water, gelatin, gum arabic, lactose, starch, magnesium stearate, talc, vegetable oils, polyalkylene glycols etc. The pharmaceutical products may be in solid form, for example as tablets, coated tablets, suppositories, capsules, in semisolid form, for example as ointments, creams, gels, suppositiories, emulsions or in liquid form, for example as solutions, suspensions or emulsions.

They comprise where appropriate excipients which are intended to act for example as fillers, binders, disintegrants, lubricants, solvents, solubilizers, masking flavors, colorant, emulsifiers. Examples of types of excipients for the purpose of the invention are saccharides (mono-, di-, tri-, oligo-, and/or polysaccharides), fats, waxes, oils, hydrocarbons, anionic, nonionic, cationic natural, synthetic or semisynthetic surfactants. They additionally comprise where appropriate excipients such as preservatives, stabilizers, wetting agents or emulsifiers; salts to modify the osmotic pressure or buffers.

The present invention likewise relates to these pharmaceutical products.

It is expedient to produce aerosol solutions for inhalation.

Suitable for oral use are in particular tablets, coated tablets or capsules with talc and/or hydrocarbon carriers or binders, such as, for example, lactose, corn starch or potato starch. Use can also take place in liquid form, such as, for example, as solution to which, where appropriate, a sweetener is added. Clathrates are likewise also suitable for oral use of such compounds, examples of clathrates which may be mentioned being those with alpha-, beta-, gamma-cyclodextrin or else beta-hydroxypropylcyclodextrin.

Sterile, injectable, aqueous or oily solutions are used for parenteral administration. Particularly suitable are injection solutions or suspensions, especially aqueous solutions of active compounds in polyethoxylated castor oil.

Examples suitable and customary for vaginal administration are pessaries, tampons or intrauterine device.

Appropriately prepared crystal suspensions can be used for intraarticular injection.

It is possible to use for intramuscular injection aqueous and oily injection solutions or suspensions and appropriate depot preparations.

For rectal administration, the novel compounds can be used in the form of suppositories, capsules, solutions (e.g. in the form of enemas) and ointments both for systemic and for local therapy.

The novel compounds can be used in the form of aerosols and inhalations for pulmonary administration.

For local use on the eyes, external auditory canal, middle ear, nasal cavity and paranasal sinuses, the novel compounds can be used as drops, ointments and tinctures in appropriate pharmaceutical preparations.

Formulations possible for topical application are gels, ointments, fatty ointments, creams, pastes, dusting powders, milk and tinctures. The dosage of the compounds of the general formula I should in these preparations be 0.01%-20% in order to achieve an adequate pharmacological effect.

The dosage of the active ingredients may vary depending on the route of administration, age and weight of the patient, nature and severity of the disorder to be treated and similar factors. Treatment can take place by single dosages or by a large number of dosages over a prolonged period. The daily dose is 0.5-1000 mg, preferably 50-200 mg, it being possible to give the dose as a single dose to be administered once or divided into 2 or more daily doses.

Carrier systems which can be used are also surface-active excipients such as salts of bile acids or animal or vegetable phospholipids, but also mixtures thereof, and liposomes or constituents thereof.

The present invention likewise relates to the formulations and dosage forms described above.

Administration of the compounds of the invention can take place by any conventional method, including oral and parenteral, e.g. by subcutaneous or intramuscular injections. The present invention likewise relates to enteral, parenteral, vaginal and oral administrations.

The compounds of the invention of the general formula I bind to the EP₂ receptor and have agonistic or antagonistic effect. It is possible to determine whether an agonistic or an antagonistic effect is present by an agonism test (see Example 1.2.1. of the Biological Examples) or by an antagonism test (see Example 1.2.2. of the Biological Examples).

Antagonists mean molecules which bind to their corresponding receptors and which inhibit the initiation of the signal transduction pathway(s) coupled to the receptor by the naturally occurring ligand(s). The antagonists normally compete with the naturally occurring ligand of the receptor for binding to the receptor. However, other modifications of the receptor are also possible by molecules which prevent the signal transduction pathways coupled to the receptor being activated by the naturally occurring ligand(s) (e.g. non-competitive, steric modifications of the receptor).

Receptor antagonists typically bind selectively to their particular receptor and not to other receptors. They normally have a higher binding affinity than the natural ligand. Although antagonists which have a higher affinity for the receptor than the natural ligand are preferred, it is likewise possible to employ antagonists having a lower affinity.

The antagonists preferably bind reversibly to their corresponding receptors.

The EP₂ receptor antagonist has a preferred affinity for the EP₂ receptor compared with any other EP receptor. The antagonism is measured in the presence of the natural agonist (PGE₂).

Agonists mean molecules which bind to their corresponding receptors and normally compete with the naturally occurring ligand of the receptor for binding to the receptor, and which stimulate the initiation of the signal transduction pathway coupled to the receptor. Agonists may also assist the binding of the natural ligand.

Receptor agonists typically bind selectively to their particular receptor and not to other receptors. They normally have a higher binding affinity than the natural ligand. Although agonists which have a higher affinity for the receptor than the natural ligand are preferred, it is likewise possible to employ agonists having a lower affinity.

The agonists preferably bind reversibly to their corresponding receptors.

The EP₂ receptor agonist has a preferred affinity for the EP₂ receptor compared with any other EP receptor.

Agonists are tested via the initiation of the signal transduction and/or physiological effect mediated by the corresponding receptor.

The compounds or low molecular weight substances which bind to a receptor are referred to as ligands. Their binding is normally reversible. Binding of a ligand to the corresponding receptor activates or inactivates the signal transduction pathway coupled to the receptor. The ligand mediates its intracellular effect in this manner. Ligands mean agonists and antagonists of a receptor.

The substance of Example 6 shows no inhibition in the cellular agonism test but a good activity (IC₅₀=1.3×10 E-6 M) in the antagonism test.

The present invention likewise relates to the use of the substances of the invention as EP₂ receptor antagonists for the treatment of disorders which are caused by disturbances in the signal transduction chain in which the EP₂ receptor is involved, such as, for example, pain and fertility impairments, and which are likewise suitable for controlling fertility.

The oocyte is surrounded in the preovulatory antral follicle by cumulus cells which form a dense ring of cells around the oocyte. After the lutenizing hormone peak (LH peak), a series of processes is activated and leads to a large morphological change in this ring of cells composed of cumulus cells. In this case, the cumulus cells form an extracellular matrix which leads to so-called cumulus expansion (Vanderhyden et al. Dev Biol. 1990 August; 140 (2):307-317). This cumulus expansion is an important constituent of the ovulatory process and of the subsequent possibility of fertilization.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. 1999 Aug. 31; 96 (18):10501-6) show a distinctly reduced cumulus expansion and severe subfertility, demonstrating the importance of the prostanoid EP₂ receptor for this process.

The substances of the invention have inhibitory effects in cumulus expansion tests.

The present invention relates to the use of the substances of the invention for controlling fertility.

Whereas the EP₂ receptor antagonist AH 6809 inhibits cumulus expansion by about only 30% and not until the concentration is 100-200 μM, a 30% inhibition of cumulus expansion can be achieved in the presence of the substance of Example 6 at a concentration which is 10 to 20 times lower (10 μm). In these experiments, the test substances compete with the natural EP₂ receptor agonist PGE₂.

The present invention relates to the use of the substances of the invention for inhibiting cumulus expansion and thus ovulation and fertilization for contraception.

Prostaglandins play an important part in angiogenesis (Sales, Jabbour, 2003, Reproduction 126, 559-567; Kuwano et al., 2004, FASEB J. 18, 300-310; Kamiyama et al., 2006, Oncogene 25, 7019-7028; Chang et al. 2005, Prostaglandins & other Lipid Mediators 76, 48-58).

Endometriosis is a chronic disorder caused by impairments of blood vessels. About 10% of women regularly suffer from heavy bleeding during menstruation, caused by changes in the blood vessels of the endometrium. In addition, structural differences in the blood vessels have been observed, such as, for example, incomplete formation of the smooth muscle cell layer (Abberton et al., 1999, Hum. Reprod. 14, 1072-1079). Since the blood loss during menstruation is partly controlled by constriction of the blood vessels, it is obvious that the defects in the smooth muscles make a substantial contribution to the bleeding.

The present invention relates to the use of the substances of the general formula I for treating endometriosis.

Prostaglandins play an important part in uterine contraction, and excessively strong contractions are responsible for painful menstruation (Sales, Jabbour, 2003, Reproduction 126, 559-567).

The present invention relates to the use of the substances of the general formula I for the treatment of painful menstruation.

Increasing research results also demonstrate the importance of EP receptors, and especially of the EP₂ receptor, in a large number of types of cancer (e.g. breast cancer, colon carcinoma, lung cancer, prostate cancer, leukemia, skin cancer), suggesting future possibilities of employing modulators (antagonists or agonists) of the EP₂ receptor for the therapy and prevention (prophylactic and/or adjuvant) of cancer (Fulton et al. Cancer Res 2006; 66 (20): 9794-7; Castellone et al. Science VOL 310 2005, 1504-1510; Chang et al. Cancer Res 2005; 65 (11): 4496-9); Hull et al. Mol Cancer Ther 2004; 3 (8):1031-9; Richards et al. J Clin Endocrinol Metab 88: 2810-2816, 2003; Sinha et al. 2007, Cancer Res; 67 (9):4507-13; Wang et al. 2004, Seminars in Oncology, Vol 31, No 1, Suppl 3: pp 64-73).

The present invention relates to the use of the substances of the general formula I for the treatment and prevention of cancers.

Prostaglandins also play an important part in processes counteracting osteoporosis. The present invention therefore relates to the use of the substances of the invention for the treatment of osteoporosis.

Reinold et al. (J. Clin. Invest. 115, 673-679 (2005)) describes PGE₂ receptors of the EP₂ subtype as the key signaling elements in inflammatory hyperalgesia. Mice no longer having this receptor (EP₂^{-/-}) do not experience spinal inflammatory pain. There is evidence that an inflammatory, increased pain sensitivity can be treated by targeted modulation of EP₂ receptors.

The present invention relates to the use of the substances of the invention for the treatment of inflammatory hyperalgesia.

Where the preparation of the starting compounds is not described, they are known or can be prepared in analogy to known compounds or processes described herein. It is likewise possible to carry out all the reactions described herein in parallel reactors or using combinatorial techniques.

The salts are prepared in a conventional way by mixing a solution of the compound of the formula I with the equivalent amount or an excess of a base or acid, which is in solution where appropriate, and separating off the precipitate or working up the solution in a conventional way.

The invention thus also relates to medicaments based on compounds of the general formula I and usual excipients or carriers.

The invention additionally relates to a process for preparing the compounds of the invention of the general formula I, which comprises reacting a compound of the formula II

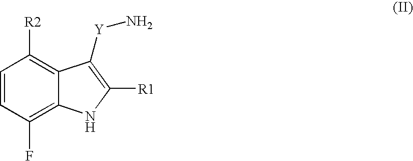

in which R¹, R² and Y have the meanings indicated above, with a carboxylic acid derivative of the general formula III

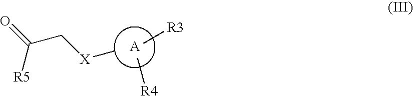

in which A, R³, R⁴ and X have the meanings indicated above, and R⁵ may be a hydroxy group, a chlorine or bromine atom or a C₁-C₆-alkyl radical, with preference for hydrogen, chlorine, the methyl or ethyl radical, by methods known to the skilled worker, and subsequently eliminating protective groups required where appropriate.

In the case where R⁵ is a hydroxy group, the reaction can initially take place by activating the acid function, and in this case for example the carboxylic acid of the formula III is initially converted in the presence of a tertiary amine such as, for example, triethylamine with isobutyl chloroformate into the mixed anhydride. Reaction of the mixed anhydride with the alkali metal salt of the appropriate amine takes place in an inert solvent or solvent mixture such as, for example, tetrahydrofuran, dimethoxyethane, dimethylformamide, hexamethylphosphoric triamide, at temperatures between −30° C. and +60° C., preferably at 0° C. to 30° C.

A further possibility is to activate the carboxylic acid by reagents such as, for example, HOBt or HATU. Reaction of the acid takes place for example with HATU in an inert solvent such as, for example, DMF in the presence of the appropriate amine of the general formula III and a tertiary amine such as, for example, ethyldiisopropylamine at temperatures between −50 and +60° C., preferably at 0° C. to 30° C.

In the case where R⁵ is C₁-C₆-alkyl it is also possible for example to carry out a direct amidolysis of the ester with the appropriate amine, possibly with the assistance of trialkylaluminum reagents, preferably trimethylaluminum.

In the case where R⁵ is a chlorine or bromine atom it is possible for example to carry out the reaction for example in pyridine or an inert solvent such as, for example, DMF in the presence of the appropriate amine of the general formula II and a tertiary amine such as, for example, ethyldiisopropylamine at temperatures between −50 and +60° C., preferably at 0° C. to 30° C.

The compounds of the general formula II which serve as starting materials are either known or can be prepared for example by reacting in a manner known per se the known hydrazines IV, where appropriate prepared from the corresponding known anilines by nitrosation followed by a reduction,

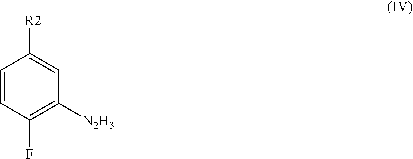

in which R² has the meaning indicated above,

a) with a ketone of the general formula V in which R¹ and Y have the meaning indicated above, and n=2 and 3, in a Fischer indole cyclization

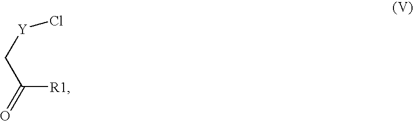

or

b) with an enol ether of the general formula VI in which R¹ and Y have the meaning indicated above, and n=2 and 3, in a Fischer indole cyclization (Org. Lett. 2004, 79ff),

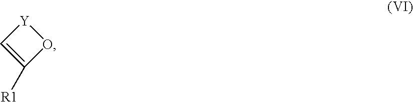

and converting the subsequently obtained alcohol by methods known to the skilled worker by conversion into a leaving group such as tosylate, mesylate, trifluoromesylate, chloride, bromide or iodide and subsequent reaction with, for example, sodium azide followed by a hydrolysis with PPh₃/H₂O in tetrahydrofuran into the amino function,

or

c) with a keto ester of the general formula VII in the case of Y with n=1

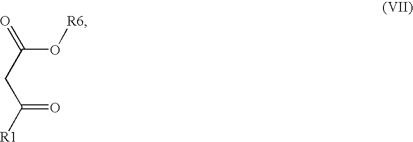

in which R¹ has the meaning indicated above, and R⁶ is a C₁-C₆-alkyl radical, in a Fischer indole cyclization, and subsequently reducing the resulting ester by methods known to the skilled worker such as, for example, diisobutylaluminum hydride in an inert solvent at temperatures between −50 and 25° C., preferably between −30 and 0° C., to the corresponding alcohol which is in turn converted into the amino function by conversion into a leaving group such as tosylate, mesylate, trifluoromesylate, chloride, bromide or iodide and subsequent reaction with, for example, sodium azide, followed by a hydrolysis with PPh₃/H₂O in tetrahydrofuran.

The invention additionally relates to an alternative process for preparing the compounds of the invention of the general formula I, which comprises firstly reacting a compound of the formula II

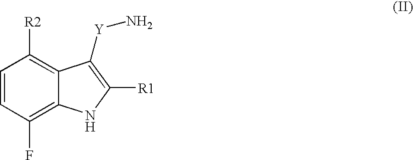

in which R¹, R² and Y have the meanings indicated above, with bromoacetyl chloride to give the compounds of the general formula VIII

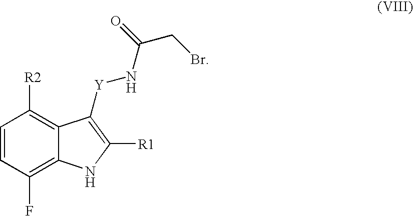

The compounds of the general formula VIII are then reacted in an etherification or N-alkylation which is known to the skilled worker, optionally with uses of bases such as, for example, potassium carbonate, cesium carbonate or diethylisopropylamine in an inert solvent, such as, for example, acetone, DMSO, DMF or acetonitrile, at temperatures between −20° C. and 100° C., 0° C. to 80° C. is preferred, with compounds of the general formula IX

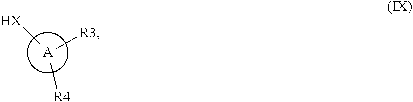

in which A, X, R³ and R⁴ have the meanings indicated above, to give the compounds of the general formula I, and protective groups which are required where appropriate are subsequently cleaved.

It is possible where appropriate for the compounds of the general formula (I) with R²═CN also to be prepared starting from the corresponding halides, preferably bromine or chlorine, by a Cu- or Pd-catalyzed (e.g. Pd(OAc)₂) cyanide introduction with Zn(CN)₂ or else K₃[Fe(CN)₆] in an inert solvent such as dimethylacetamide, dimethylformamide or N-methylpyrolidone at temperatures between 60° C. and the boiling point of the respective solvent.

### Preparation of the Compounds of the Invention

The following examples illustrate the preparation of the compounds of the invention of the general formula (I) without restricting the scope of the claimed compounds to these examples.

The compounds of the invention of the general formula (I) can be prepared as described below.

### EXAMPLE 1

### N-[2-(7-Fluoro-2,4-dimethyl-1H-indol-3-yl)ethyl]2-(4-methoxyphenoxy)acetamide

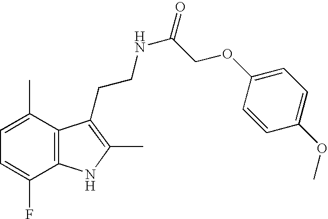

149 mg of cesium carbonate are added to a solution of 120 mg of the bromide prepared in example 1a) and 45.5 mg of 4-methoxyphenol in 3.6 ml of dimethylformamide, and the mixture is stirred at 75° C. for 20 hours. Cooling is followed by dilution with ethyl acetate, and the organic phase is washed once with water, dried over sodium sulfate and, after filtration, concentrated in vacuo. The residue obtained in this way is purified by chromatography on silica gel with hexane/0-100% ethyl acetate. 53 mg of the title compound are obtained in this way.

NMR (300 MHz, DMSO-d6): δ=2.29 (3H), 2.55 (3H), 2.86 (2H), 3.21 (2H), 3.66 (3H), 4.35 (2H), 6.55 (1H), 6.61 (1H), 6.83 (2H), 6.87 (2H), 8.17 (1H), 11.13 (1H).

The starting material for the above title compound is prepared as follows:

### 1a) 2-Bromo-N-[2-(7-fluoro-2,4-dimethyl-1H-indol-3-yl)ethyl]acetamide

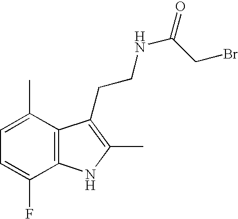

1.43 ml of triethylamine are added to a solution of 1.0 g of 2-(7-fluoro-2,4-dimethyl-1H-indol-3-yl)ethylamine hydrochloride in 30 ml of dimethylformamide and the mixture is stirred at 25° C. for 10 minutes. Then, at this temperature, 0.34 ml of bromacetyl chloride is added dropwise, followed by further stirring at 25° C. for 45 minutes.

The reaction solution is then added to ice-water and extracted twice with ethyl acetate. The combined organic phases are washed twice with water, dried over sodium sulfate and, after filtration, are concentrated in vacuo. The residue obtained in this way is purified by chromatography on silica gel with hexane/0-100% ethyl acetate. The yield is 916 mg of the title compound.

NMR (300 MHz, DMSO-d6): δ=2.28 (3H), 2.53 (3H), 2.84 (2H), 3.16 (2H), 3.82/4.03 (2H), 6.50-6.67 (2H), 8.34/8.40 (1H), 11.13 (1H).

### EXAMPLE 2

### N-[2-(7-Fluoro-2,4-dimethyl-1H-indol-3-yl)ethyl]-2-(3-methoxy-phenoxy)acetamide

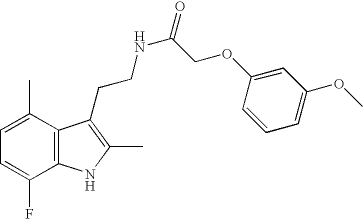

In analogy to example 1, the yield from 100 mg of the bromide from example 1a) and 0.034 ml of 3-methoxyphenol is 65.4 mg of the title compound.

NMR (300 MHz, DMSO-d6): δ=2.29 (3H), 2.55 (3H), 2.87 (2H), 3.21 (2H), 3.70 (3H), 4.41 (2H), 6.48-6.65 (5H), 7.16 (1H), 8.20 (1H), 11.13 (1H).

### EXAMPLE 3

### N-[2-(7-Fluoro-2,4-dimethyl-1H-indol-3-yl)ethyl]-2-(2-methoxy-phenoxy)acetamide

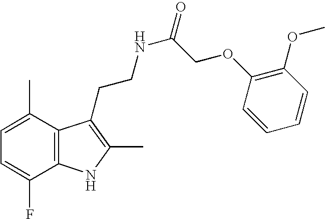

In analogy to example 1, the yield from 100 mg of the bromide from example 1a) and 37.9 mg of 2-methoxyphenol is 53.6 mg of the title compound.

NMR (300 MHz, DMSO-d6): δ=2.28 (3H), 2.54 (3H), 2.86 (2H), 3.23 (2H), 3.72 (3H), 4.40 (2H), 6.51-6.60 (2H), 6.78-7.01 (4H), 8.01 (1H), 11.12 (1H).

### EXAMPLE 4

### N-[2-(7-Fluoro-2,4-dimethyl-1H-indol-3-yl)ethyl]-2-(4-fluorophenyl-thio)acetamide

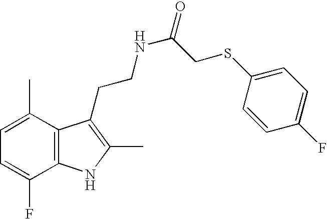

259 mg of N-[(dimethylamino)-1H-1,2,3-triazolo[4,5-b]pyridin-1-ylmethylene]-N-methylmethanaminium hexafluorophosphate N-oxide (HATU) and 100 mg of 2-(7-fluoro-2,4-dimethyl-1H-indol-3-yl)ethylamine hydrochloride are added to a solution of 84.4 mg of 2-(4-fluorophenylthio)acetic acid in 3 ml of dimethylformamide. Then, at 0° C., 0.15 ml of ethyldiisopropylamine is added dropwise, and the mixture is stirred at 25° C. for 20 hours. Then 40 ml of a mixture of ice and conc. aqueous bicarbonate solution are added, followed by extraction three times with ethyl acetate. The combined organic phases are washed once with saturated sodium chloride solution, dried over sodium sulfate and, after filtration, concentrated in vacuo. The residue obtained in this way is purified by chromatography on silica gel with hexane/0-100% ethyl acetate. The yield is 85.5 mg of the title compound.

NMR (300 MHz, DMSO-d6): δ=2.26 (3H), 2.50 (3H), 2.77 (2H), 3.11 (2H), 3.56 (2H), 6.51-6.65 (2H), 7.13 (2H), 7.38 (2H), 8.21 (1H), 11.12 (1H).

The following examples are prepared in analogy to example 1 and purified by HPLC:

### HPLC Method:

Instrument: analytical 4-channel MUX system with CTC Pal injector, Waters 1525 pumps, Waters 2488 UV detector and Waters ZQ 2000 single quad MS detector.
Column X-Bridge RP C18 4.6×50 3.5 μm; detection wavelength 214 nm; flow rate 2 ml/min; eluents A: 0.1% TFA in H₂O, B 0.1% TFA in ACN; gradient in each case based on B: 1% to 99% (5′) to 99% (1′) to 1% (0.25′) to 1% (1.75′)

| | | | Theoret- | | |
|---|---|---|---|---|---|
| | | | ical | | |
| Ex- | | | mass | Mass | Retention |
| am- | | | m/z | found m/z | time |
| ple | Structure | Name | [M + H]⁺ | [M + H]+ | [min.] |
| 5 | 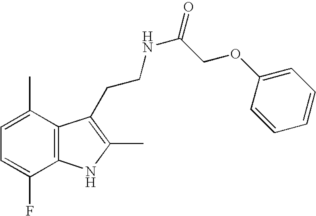 | N-[2-(7-Fluoro-2,4-dimethyl-1H-indol-3-yl)-ethyl]-2-phenoxy-acetamide | 341 | 341 | 9.24 |
| 6 | 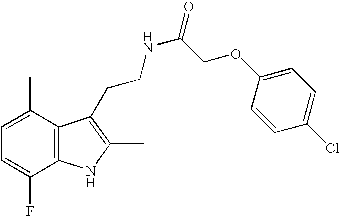 | 2-(4-Chlorophenoxy)-N-[2-(7-fluoro-2,4-dimethyl-1H-indol-3-yl)ethyl]-acetamide | 375 | 375 | 9.87 |
| 7 | 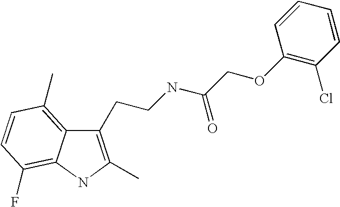 | 2-(2-Chlorophenoxy)-N-[2-(7-fluoro-2,4-dimethyl-1H-indol-3-yl)ethyl]-acetamide | 375 | 376 | 5.09 |
| 8 | 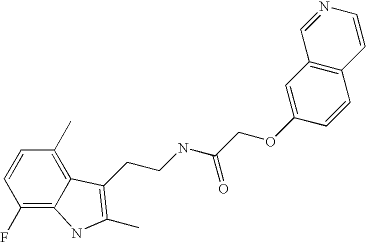 | N-[2-(7-Fluoro-2,4-dimethyl-1H-indol-3-yl)-ethy]-2-(isoquinolin-7-yloxy)acetamide | 391 | 392 | 2.95 |
| 9 | 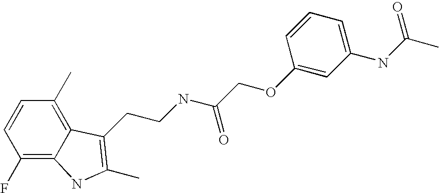 | 2-(3-Acetylamino-phenoxy)-N-[2-(7-fluoro-2,4-dimethyl-1H-indol-3-yl)ethyl]acetamide | 397 | 398 | 3.41 |
| 10 | 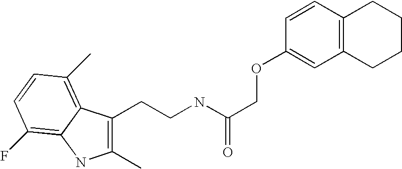 | N-[2-(7-Fluoro-2,4-dimethyl-1H-indol-3-yl)-ethyl]-2-(5,6,7,8-tetrahydronaphthalen-2-yloxy)acetamide | 394 | 395 | 4.59 |
| 11 | 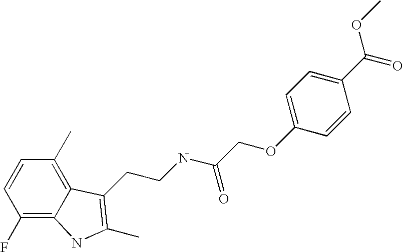 | 4-{[2-(7-Fluoro-2,4-dimethyl-1H-indol-3-yl)-ethylcarbamoyl]-methoxy} benzoic acidmethyl ester | 398 | 399 | 3.9 |
| 12 | 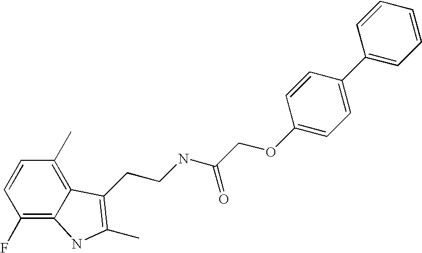 | 2-(Biphenyl-4-yloxy)-N-[2-(7-fluoro-2,4-dimethyl-1H-indol-3-yl)-ethyl]-acetamide | 416 | 417 | 4.46 |
| 13 | 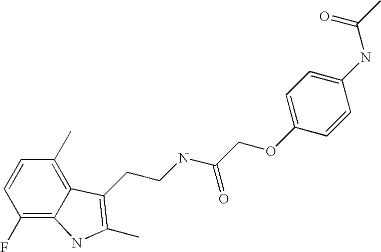 | 2-(4-Acetylamino-phenoxy)-N-[2-(7-fluoro-2,4-dimethyl-1H-indol-3-yl)ethyl]acetamide | 397 | 398 | 3.39 |
| 14 | 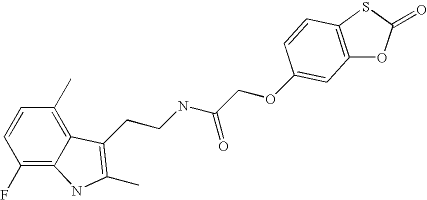 | N-[2-(7-Fluoro-2,4-dimethyl-1H-indol-3-yl)-ethyl]-2-(2-oxo-benzo[1,3]oxathiol-6-yloxy]acetamide | 414 | 415 | 4.03 |
| 15 | 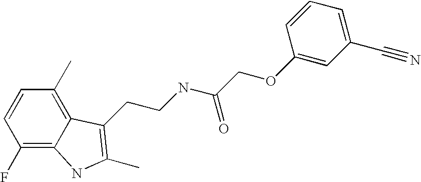 | 2-(3-Cyanophenoxy)-N-[2-(7-fluoro-2,4-dimethyl-1H-indoi-3-yl)ethyl]-acetamide | 365 | 366 | 3.89 |
| 16 | 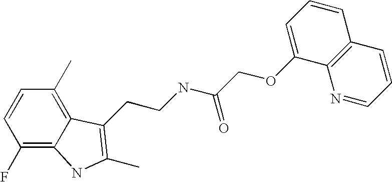 | N-[2-(7-Fluoro-2,4-dimethyl-1H-indol-3-yl)-ethyl]-2-(quinolin-8-yloxy)acetamide | 391 | 392 | 2.96 |
| 17 | 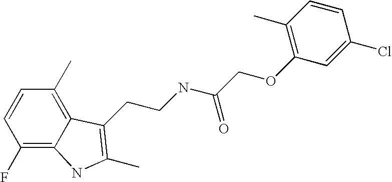 | 2-(5-Chloro-2-methyl-phenoxy)-N-[2-(7-fluoro-2,4-dimethyl-1H-indol-3-yl)ethyl]acetamide | 389 | 390 | 4.37 |
| 18 | 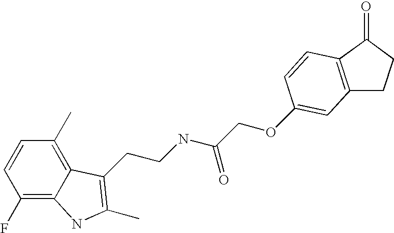 | N-[2-(7-Fluoro-2,4-dimethyl-1H-indol-3-yl)-ethyl]-2-(1-oxoindan-5-yloxy)acetamide | 394 | 395 | 3.64 |
| 19 | 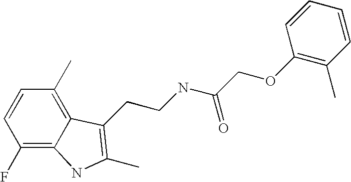 | N-[2-(7-Fluoro-2,4-dimethyl-1H-indol-3-yl)-ethyl]-2-o-tolyloxy-acetamide | 354 | 355 | 4.13 |
| 20 | 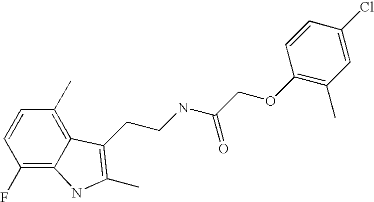 | 2-(4-Chloro-2-methyl-phenoxy)-N-[2-(7-fluoro-2,4-dimethyl-1H-indol-3-yl)ethyl]acetamide | 389 | 390 | 4.44 |
| 21 | 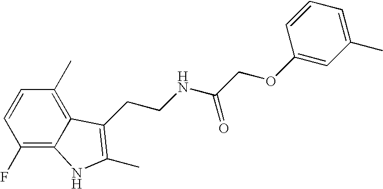 | N-[2-(7-Fluoro-2,4-imethyl-1H-indol-3-yl)ethyl]-2-m-tolyloxyacetamide | 354 | 355 | 4.18 |
| 22 | 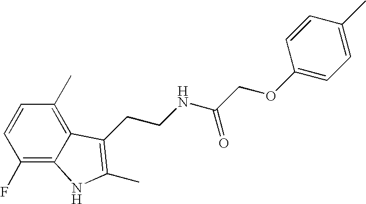 | N-[2-(7-Fluoro-2,4-imethyl-1H-indol-3-yl)ethyl]-2-p-tolyloxyacetamide | 354 | 355 | 4.18 |
| 23 | 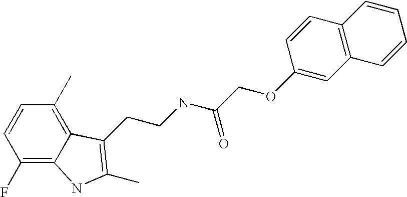 | N-[2-(7-Fluoro-2,4-dimethyl-1H-indol-3-yl)-ethyl]-2-(naphthalen-2-yloxy)acetamide | 390 | 391 | 4.28 |
| 24 | 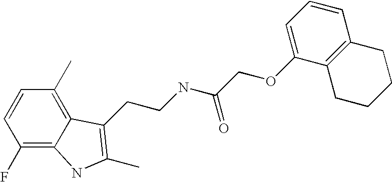 | N-[2-(7-Fluoro-2,4-dimethyl-1H-indol-3-yl)-ethyl]-2-(5,6,7,8-tetrahydronaphthalen-1-yloxy)acetamide | 394 | 395 | 4.61 |
| 25 | 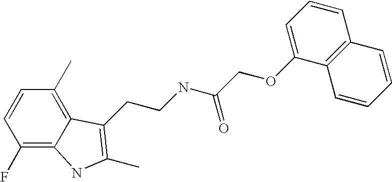 | N-[2-(7-Fluoro-2,4-dimethyl-1H-indol-3-yl)-ethyl]-2-(naphthalen-1-yloxy)acetamide | 390 | 391 | 4.35 |
| 26 | 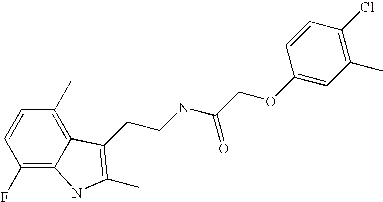 | 2-(4-Chloro-3-methyl-phenoxy)-N-[2-(7-fluoro-2,4-dimethyl-1H-indol-3-yl)ethyl]acetamide | 389 | 390 | 4.41 |
| 27 | 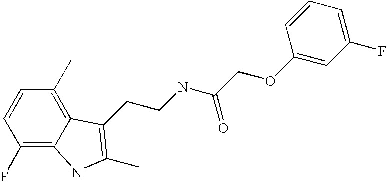 | N-[2-(7-Fluoro-2,4-dimethyl-1H-indol-3-yl)-ethyl]-2-(3-fluoro-phenoxy)acetamide | 358 | 359 | 4.05 |
| 28 | 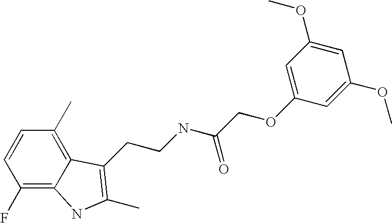 | 2-(3,5-Dimethoxy-phenoxy)-N-[2-(7-fluoro-2,4-dimethyl-1H-indol-3-yl)ethyl]acetamide | 400 | 401 | 3.97 |
| 29 | 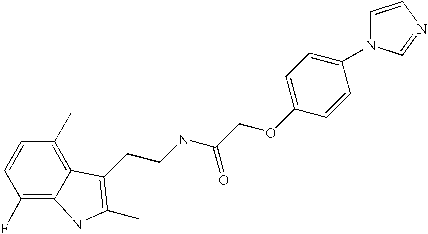 | N-[2-(7-Fluoro-2,4-dimethyl-1H-indol-3-yl)-ethyl]-2-(4-imidazol-1-yl-phenoxy)acetamide | 406 | 407 | 2.94 |
| 30 | 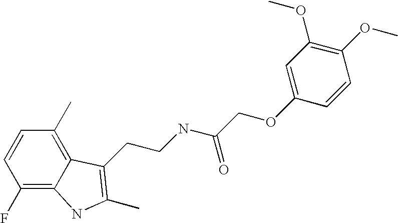 | 2-(3,4-Dimethoxy-phenoxy)-N-[2-(7-fluoro-2,4-dimethyl-1H-indol-3-yl)ethyl]acetamide | 400 | 401 | 3.8 |
| 31 | 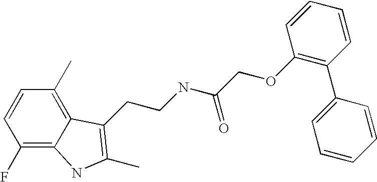 | 2-(Biphenyl-2-yloxy)-N-[2-(7-fluoro-2,4-dimethyl-1H-indol-3-yl)ethyl]acetamide | 416 | 417 | 4.59 |
| 32 | 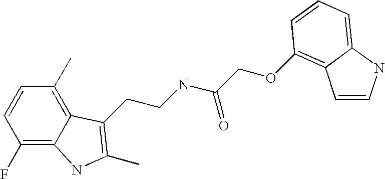 | N-[2-(7-Fluoro-2,4-dimethyl-1H-indol-3-yl)-ethyl]-2-(1H-indol-4-yloxy)acetamide | 379 | 380 | 3.75 |
| 33 | 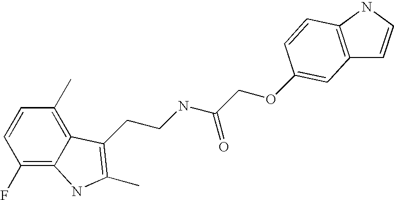 | N-[2-(7-Fluoro-2,4-dimethyl-1H-indol-3-yl)-ethyl]-2-(1H-indol-5-yloxy)acetamide | 379 | 380 | 3.83 |
| 34 | 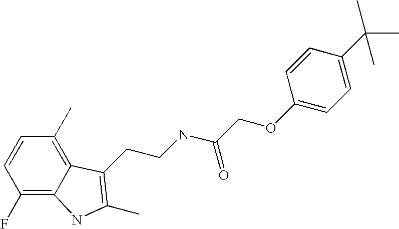 | 2-(4-tert-Butylphenoxy)-N-[2-(7-fluoro-2,4-dimethyl-1H-indol-3-yl)-ethyl]acetamide | 397 | 398 | 4.67 |
| 35 | 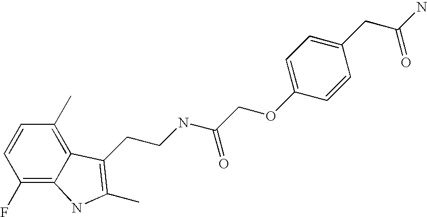 | 2-(4-{[2-(7-Fluoro-2,4-dimethyl-1H-indol-3-yl)-ethylcarbamoyl]-methoxy}phenyl)-acetamide | 397 | 398 | 3.28 |
| 36 | 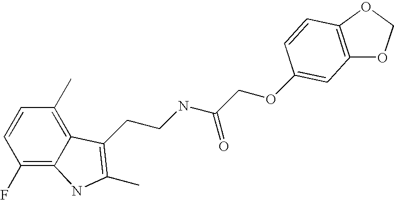 | 2-(Benzo[1,3]dioxol-5-yloxy)-N-[2-(7-fluoro-2,4-dimethyl-1H-indol-3-yl)-ethyl]acetamide | 384 | 385 | 3.94 |
| 37 | 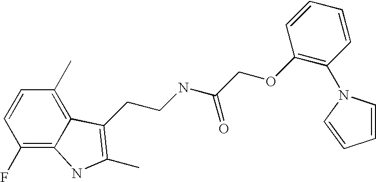 | N-[2-(7-Fluoro-2,4-dimethyl-1H-indol-3-yl)-ethyl]-2-(2-pyrrol-1-yl-phenoxy)acetamide | 405 | 406 | 4.3 |
| 38 | 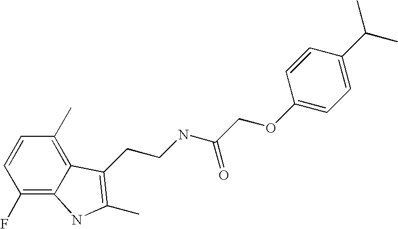 | N-[2-(7-Fluoro-2,4-dimethyl-1H-indol-3-yl)-ethyl]-2-(4-isopropyl-phenoxy)acetamide | 382 | 383 | 4.54 |
| 39 | 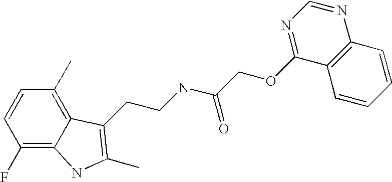 | N-[2-(7-Fluoro-2,4-dimethyl-1H-indol-3-yl)-ethyl]-2-(quinazolin-4-yloxy)acetamide | 392 | 393 | 3.35 |
| 40 | 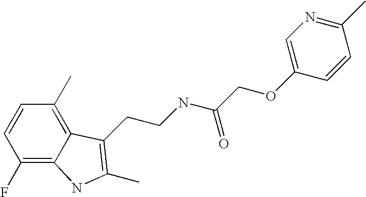 | N-[2-(7-Fluoro-2,4-dimethyl-1H-indol-3-yl)-ethyl]-2-(6-methylpyridin-3-yloxy)acetamide | 355 | 356 | 2.78 |
| 41 | 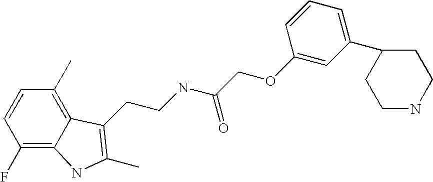 | N-[2-(7-Fluoro-2,4-dimethyl-1H-indol-3-yl)-ethyl]-2-(3-piperidin-4-yl-phenoxy)acetamide | 424 | 425 | 2.84 |
| 42 | 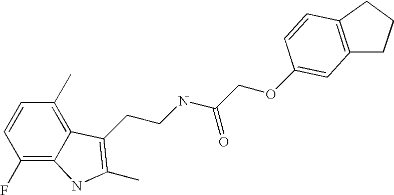 | N-[2-(7-Fluoro-2,4-dimethyl-1H-indol-3-yl)-ethyl]-2-(indan-5-yloxy)-acetamide | 380 | 381 | 4.43 |
| 43 | 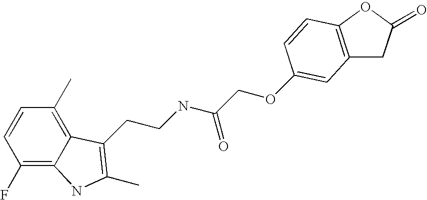 | N-[2-(7-Fluoro-2,4-dimethyl-1H-indol-3-yl)-ethyl]-2-(2-oxo-2,3-dihydrobenzofuran-5-yloxy)acetamide | 396 | 397 | 3.52 |
| 44 | 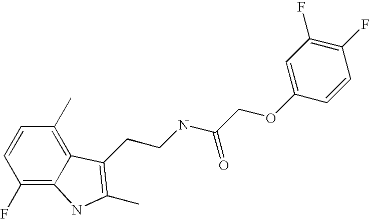 | 2-(3,4-Difluorophenoxy)-N-[2-(7-fluoro-2,4-dimethyl-1H-indol-3-yl)-ethyl]acetamide | 376 | 377 | 4.07 |
| 45 | 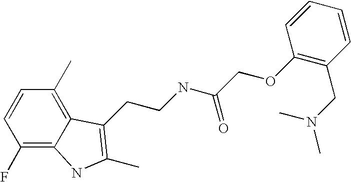 | 2-(2-Dimethylaminomethyl-phenoxy)-N-[2-(7-fluoro-2,4-dimethyl-1H-indol-3-yl)ethyl]acetamide | 397 | 398 | 3.09 |
| 46 | 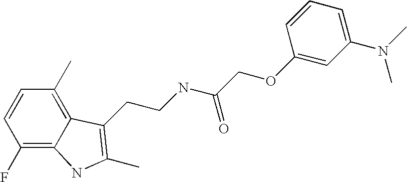 | 2-(3-Dimethylamino-phenoxy)-N-[2-(7-fluoro-2,4-dimethyl-1H-indol-3-yl)ethyl]acetamide | 383 | 384 | 3.21 |
| 47 | 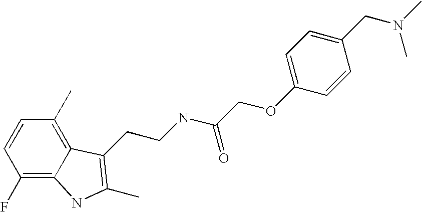 | 2-(4-Dimethylaminomethyl-phenoxy)-N-[2-(7-fluoro-2,4-dimethyl-1H-indol-3-yl)ethyl]acetamide | 397 | 398 | 2.95 |
| 48 | 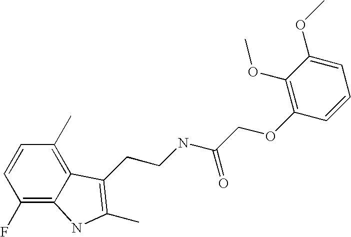 | 2-(2,3-Dimethoxy-phenoxy)-N-[2-(7-fluoro-2,4-dimethyl-1H-indol-3-yl)ethyl]acetamide | 400 | 401 | 3.83 |
| 49 | 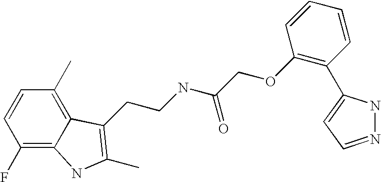 | N-[2-(7-Fluoro-2,4-dimethyl-1H-indol-3-yl)-ethyl]-2-[2-(2H-pyrazol-3-yl)phenoxy]acetamide | 406 | 407 | 4.08 |
| 50 | 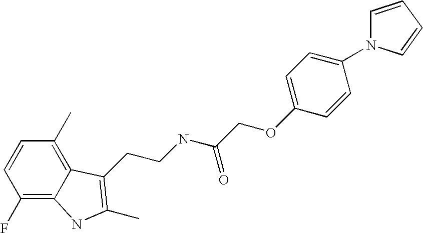 | N-[2-(7-Fluoro-2,4-dimethyl-1H-indol-3-yl)-ethyl]-2-(4-pyrrol-1-yl-phenoxy)acetamide | 405 | 406 | 4.29 |
| 51 | 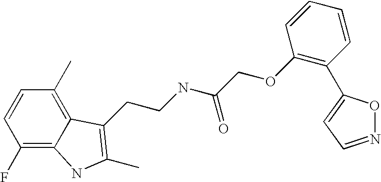 | N-[2-(7-Fluoro-2,4-dimethyl-1H-indol-3-yl)-ethyl]-2-(2-isoxazol-5-yl-phenoxy)acetamide | 407 | 408 | 3.82 |
| 52 | 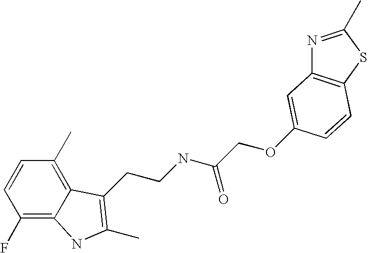 | N-[2-(7-Fluoro-2,4-dimethyl-1H-indol-3-yl)-ethyl]-2-(2-methyl-benzothiazol-5-yloxy)-acetamide | 411 | 412 | 3.82 |
| 53 | 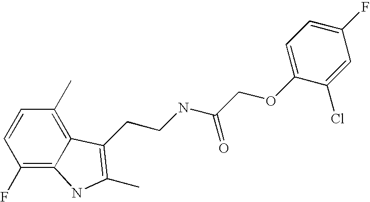 | 2-(2-Chloro-4-fluoro-phenoxy)-N-[2-(7-fluoro-2,4-dimethyl-1H-indol-3-yl)ethyl]acetamide | 393 | 394 | 4.26 |
| 54 | 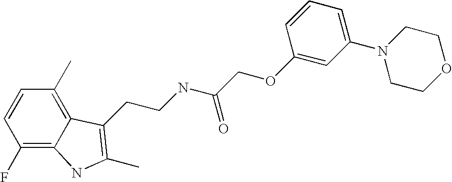 | N-[2-(7-Fluoro-2,4-dimethyl-1H-indol-3-yl)-ethyl]-2-(3-morpholin-4-ylphenoxy)acetamide | 426 | 427 | 3.7 |
| 55 | 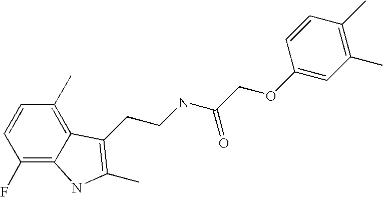 | 2-(3,4-Dimethyl-phenoxy)-N-[2-(7-fluoro-2,4-dimethyl-1H-indol-3-yl)ethyl]acetamide | 368 | 369 | 4.38 |
| 56 | 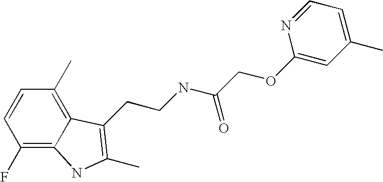 | N-[2-(7-Fluoro-2,4-dimethyl-1H-indol-3-yl)-ethyl]-2-(4-methylpyridin-2-yloxy)acetamide | 355 | 356 | 3.19 |
| 57 | 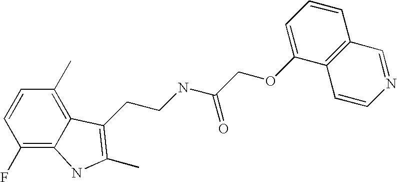 | N-[2-(7-Fluoro-2,4-dimethyl-1H-indol-3-yl)-ethyl]-2-(isoquinolin-5-yloxy)acetamide | 391 | 392 | 2.91 |
| 58 | 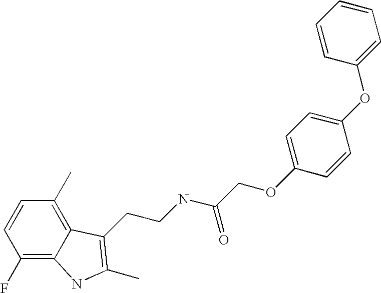 | N-[2-(7-Fluoro-2,4-dimethyl-1H-indol-3-yl)-ethyl]-2-(4-phenoxy-phenoxy)acetamide | 432 | 433 | 4.52 |
| 59 | 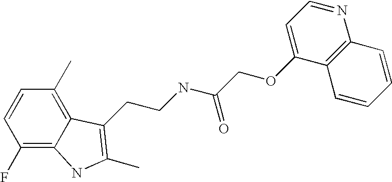 | N-[2-(7-Fluoro-2,4-dimethyl-1H-indol-3-yl)-ethyl]-2-(quinolin-4-yloxy)acetamide | 391 | 392 | 3.1 |
| 60 | 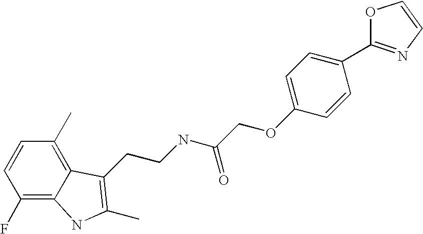 | N-[2-(7-Fluoro-2,4-dimethyl-1H-indol-3-yl)-ethyl]-2-(4-oxazol-2-yl-phenoxy)acetamide | 407 | 408 | 3.95 |
| 61 | 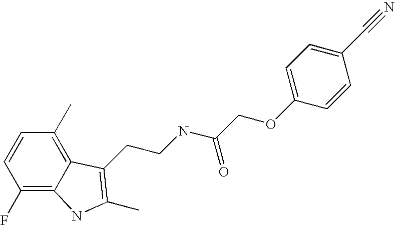 | 2-(4-Cyano-phenoxy)-N-[2-(7-fluoro-2,4-dimethyl-1H-indol-3-yl)ethyl]-acetamide | 365 | 366 | 4.07 |
| 62 | 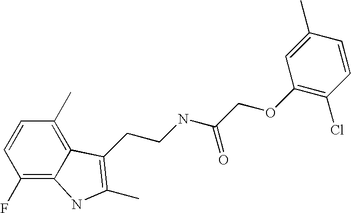 | 2-(2-Chloro-5-methyl-phenoxy)-N-[2-(7-fluoro-2,4-dimethyl-1H-indol-3-yl)ethyl]acetamide | 389 | 390 | 4.32 |
| 63 | 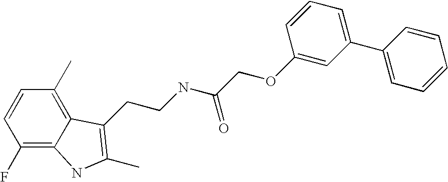 | 2-(Biphenyl-3-yloxy)-N-[2-(7-fluoro-2,4-dimethyl-1H-indol-3-yl)ethyl]-acetemide | 416 | 417 | 4.52 |
| 64 | 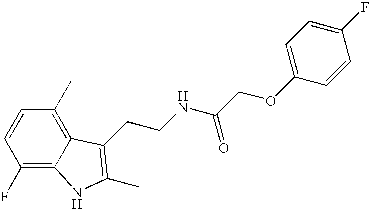 | N-[2-(7-Fluoro-2,4-dimethyl-1H-indol-3-yl)-ethyl]-2-(4-fluoro-phenoxy)acetamide | 358 | 359 | 3.95 |
| 65 | 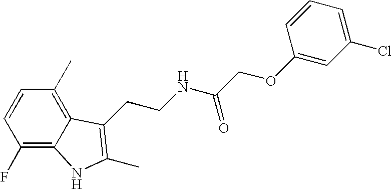 | 2-(3-Chlorophenoxy)-N-[2-(7-fluoro-2,4-dimethyl-1H-indol-3-yl)ethyl]-acetamide | 375 | 376 | 4.24 |
| 66 | 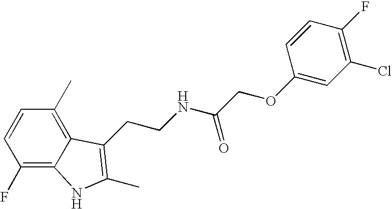 | 2-(3-Chloro-4-fluoro-phenoxy)-N-[2-(7-fluoro-2,4-dimethyl-1H-indol-3-yl)ethyl]acetamide | 393 | 394 | 4.24 |
| 67 | 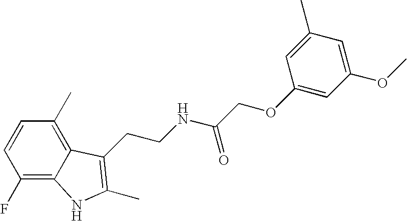 | N-[2-(7-Fluoro-2,4-dimethyl-1H-indol-3-yl)-ethyl]-2-(3-methoxy-5-methylphenoxy)-acetamide | 384 | 385 | 4.15 |
| 68 | 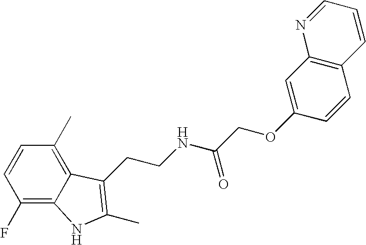 | N-[2-(7-Fluoro-2,4-dimethyl-1H-indol-3-yl)-ethyl˜2-(quinolin-7-yloxy)acetamide | 391 | 392 | 2.93 |
| 69 | 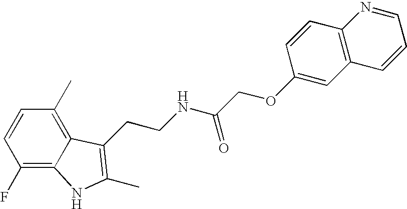 | N-[2-(7-Fluoro-2,4-dimethyl-1H-indol-3-yl)-ethyl]-2-(quinolin-6-yloxy)acetamide | 391 | 392 | 2.93 |
| 70 | 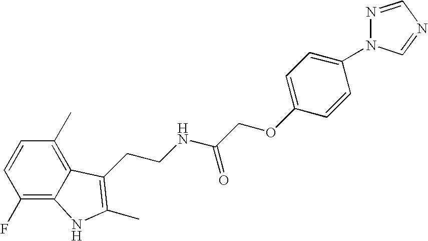 | N-[2-(7-Fluoro-2,4-dimethyl-1H-indol-3-yl)-ethyl]-2-(4-[1,2,4]triazol-1-ylphenoxy)acetamide | 407 | 408 | 3.4 |
| 71 | 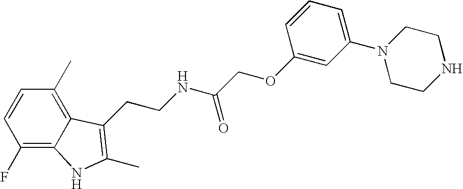 | N-[2-(7-Fluoro-2,4-dimethyl-1H-indol-3-yl)-ethyl]-2-(3-piperazin-1-yl-phenoxy)acetamide | 425 | 426 | 2.99 |
| 72 | 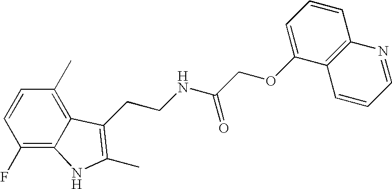 | N-[2-(7-Fluoro-2,4-dimethyl-1H-indol-3-yl)-ethyl]-2-(3-piperazin-1-yl-phenoxy)acetamide | 391 | 392 | 2.82 |
| 73 | 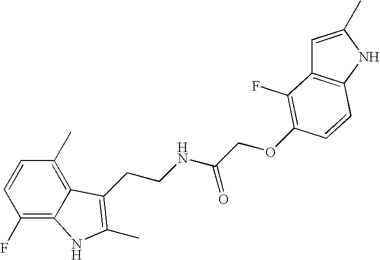 | N-[2-(7-Fluoro-2,4-dimethyl-1H-indol-3-yl)-ethyl]-2-(4-fluoro-2-methyl-1H-indol-5-yloxy)-acetemide | 411 | 412 | 4.03 |
| 74 | 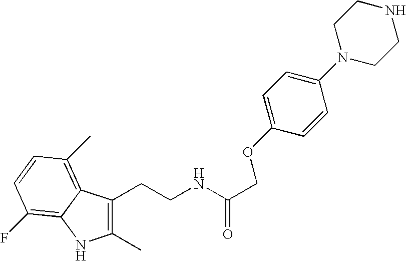 | N-[2-(7-Fluoro-2,4-dimethyl-1H-indol-3-yl)-ethyl]-2-(4-piperazin-1-yl-phenoxy)acetamide | 425 | 426 | 2.87 |
| 75 | 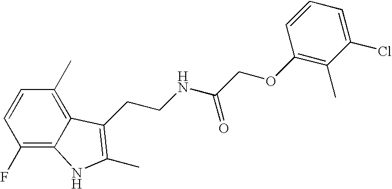 | 2-(3-Chloro-2-methyl-phenoxy)-N-[2-(7-fluoro-2,4-dimethyl-1H-indol-3-yl)ethyl]acetamide | 389 | 390 | 4.46 |
| 76 | 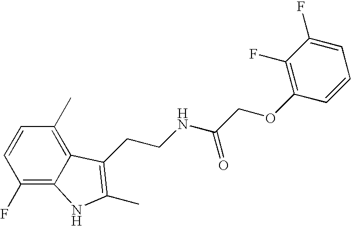 | 2-(2,3-Difluorophenoxy)-N-[2-(7-fluoro-2,4-dimethyl-1H-indol-3-yl)-ethyl]acetamide | 376 | 377 | 4.09 |
| 77 | 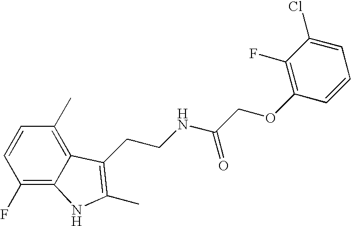 | 2-(3-Chloro-2-fluoro-phenoxy)-N-[2-(7-fluoro-2,4-dimethyl-1H-indol-3-yl)ethyl]acetamide | 393 | 394 | 4.25 |
| 78 | 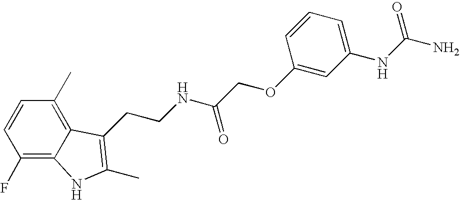 | N-[2-(7-Fluoro-2,4-dimethyl-1H-indol-3-yl)-ethyl]-2-(3-carbamoyl-aminophenoxy)acetamide | 398 | 399 | 3.23 |
| 79 | 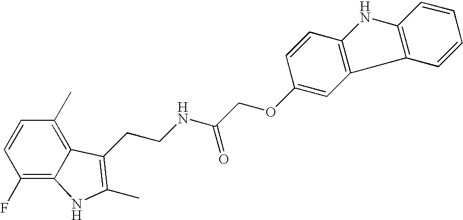 | 2-(9H-Carbazol-3-yloxy)-N-[2-(7-fluoro-2,4-dimethyl-1H-indol-3-yl)-ethyl]acetamide | 429 | 430 | 4.18 |
| 80 | 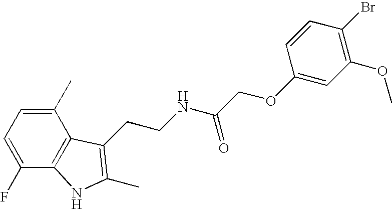 | 2-(4-Bromo-3-methoxy-phenoxy)-N-[2-(7-fluoro-2,4-dimethyl-1H-indol-3-yl)ethyl]acetamide | 449 | 450 | 4.26 |
| 81 | 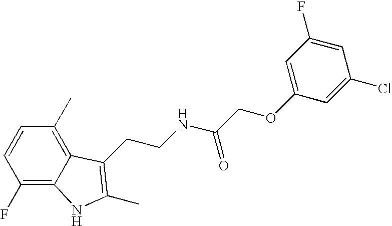 | 2-(3-Chloro-5-fluoro-phenoxy)-N-[2-(7-fluoro-2,4-dimethyl-1H-indol-3-yl)ethyl]acetamide | 393 | 394 | 4.34 |
| 82 | 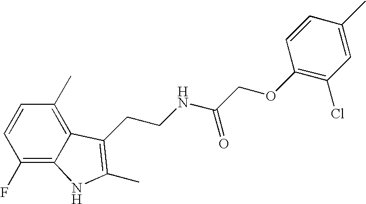 | 2-(2-Chloro-4-methyl-phenoxy)-N-[2-(7-fluoro-2,4-dimethyl-1H-indol-3-yl)ethyl]acetamide | 389 | 390 | 4.41 |
| 83 | 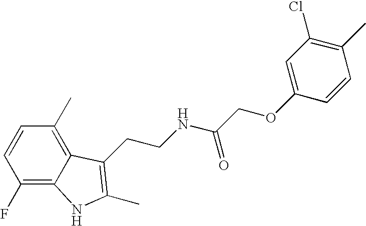 | 2-(3-Chloro-4-methyl-phenoxy)-N-[2-(7-fluoro-2,4-dimethyl-1H-indol-3-yl)ethyl]acetamide | 389 | 390 | 4.38 |
| 84 | 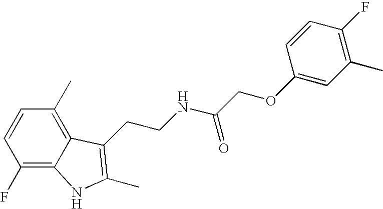 | N-[2-(7-Fluoro-2,4-dimethyl-1H-indol-3-yl)-ethyl]-2-(4-fluoro-3-methylphonoxy)-acetamide | 372 | 373 | 4.18 |
| 85 | 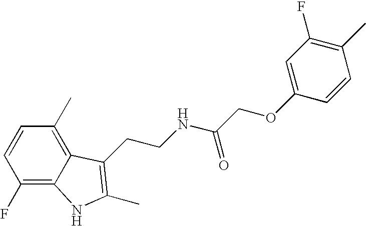 | N-[2-(7-Fluoro-2,4-dimethyl-1H-indol-3-yl)-ethyl]-2-(3-fluoro-4-methyl-phenoxy)acetamide | 372 | 373 | 4.42 |
| 86 | 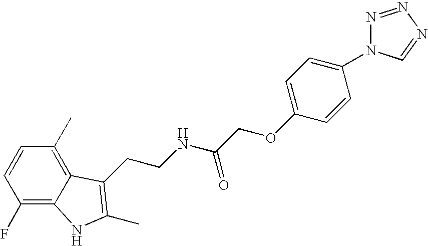 | N-[2-(7-Fluoro-2,4-dimethyl-1H-indol-3-yl)-ethyl]-2-(4-tetrazol-1-yl-phenoxy)acetamide | 408 | 409 | 3.5 |
| 87 | 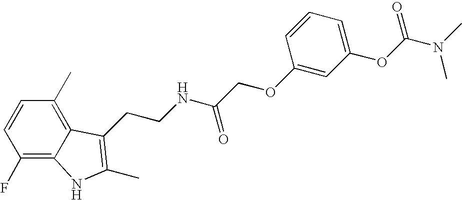 | N-[2-(7-Fluoro-2,4-dimethyl-1H-indol-3-yl)-ethyl]-2-[(3-dimethylcarbamoyl)oxy]-phenoxy)acetamide | 427 | 428 | 3.68 |
| 88 | 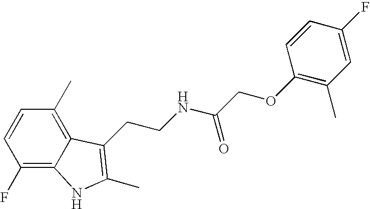 | N-[2-(7-Fluoro-2,4-dimethyl-1H-indol-3-yl)-ethyl]-2-(4-fluoro-2-methyl-phenoxy)acetamide | 372 | 373 | 4.21 |
| 89 | 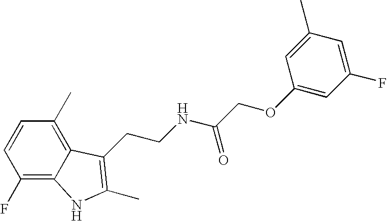 | N-[2-(7-Fluoro-2,4-dimethyl-1H-indol-3-yl)-ethyl]-2-(3-fluoro-5-methyl-phenoxy)acetamide | 372 | 373 | 4.24 |
| 90 | 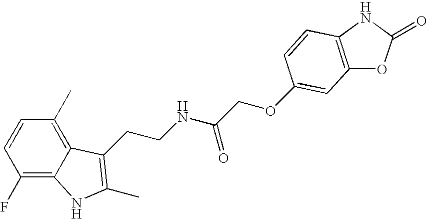 | N-[2-(7-Fluoro-2,4-dimethyl-1H-indol-3-yl)-ethyl]-2-(2-oxo-2,3-dihydrobenzooxazol-6-yloxy)acetamide | 397 | 398 | 3.35 |
| 91 | 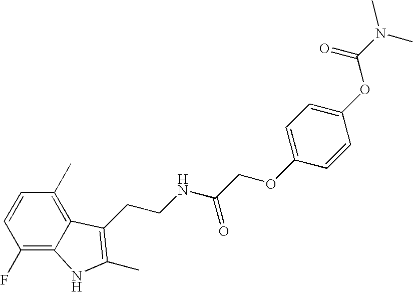 | N-[2-(7-Fluoro-2,4-dimethyl-1H-indol-3-yl)-ethyl]-2-[(4-dimethylcarbamoyl)oxy]-phenoxy)acetamide | 427 | 428 | 3.92 |
| 92 | 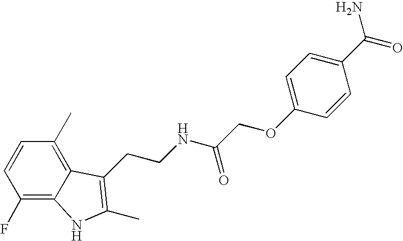 | 4-{[2-(7-Fluoro-2,4-dimethyl-1H-indol-3-yl)-ethylcarbamoyl]-methoxy}benzamide | 383 | 384 | 3.24 |
| 93 | 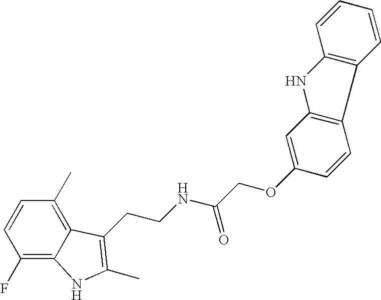 | 2-(9H-Carbazol-2-yloxy)-N-[2-(7-fluoro-2,4-dimethyl-1H-indol-3-yl)-ethyl]acetamide | 429 | 430 | 4.29 |

### Biological Examples

### 1. Detection of the Antagonism of the Human Prostaglandin E₂ (Subtype EP₂) Receptor Signal

### 1.1 Principle of Detection

The binding of PGE2 to the EP₂ subtype of the human PGE2 receptor induces activation of membrane-associated adenylate cyclases and leads to the formation of cAMP. In the presence of the phosphodiesterase inhibitor IBMX, cAMP which has accumulated due to this stimulation and been released by cell lysis is employed in a competitive detection method. In this assay, the cAMP in the lysate competes with cAMP-XL665 for binding of an Eu cryptate-labelled anti-cAMP antibody.

This results, in the absence of cellular cAMP, in a maximum signal which derives from coupling of this antibody to the cAMP-XL665 molecule. After excitation at 337 nm, this results in a FRET (fluorescence resonance energy transfer)-based, long-lived emission signal at 665 nm (and at 620 nM). The two signals are measured in a suitable measuring instrument with a time lag, i.e. after the background fluorescence has declined. Any increase in the low FRET signal caused by prostaglandin E₂ addition (measured as well ratio change=emission_{665 nm}/emission_{620 nm}*10 000) shows the effect of antagonists.

### 1.2. Detection Method

### 1.2.1 Antagonism Assay (Data for Each Well of a 384-Well Plate):

The substance solutions (0.75 μl) introduced into an assay plate and 30% DMSO are dissolved in 16 μl of a KRSB+IBMX stimulation solution (1× Krebs-Ringer Bicarbonate Buffer; Sigma-Aldrich # K-4002; including 750 μM 3-isobutyl-1-methylxanthine Sigma-Aldrich # I-7018), and then 15 μl thereof are transferred into a media-free cell culture plate which has been washed with KRSB shortly beforehand.

After preincubation at room temperature (RT) for 30 minutes, 5 μl of a 4×PGE2 solution (11 nM) are added, and incubation is carried out in the presence of the agonist at RT for a further 60 min (volume: ˜20 μl) before the reaction is then stopped by adding 5 μl of lysis buffer and incubated at RT for a further 20 min (volume: ˜25 μl). The cell lysate is then transferred into a measuring plate and measured in accordance with the manufacturer's information (cyclic AMP kit Cisbio International #62AMPPEC).

### 1.2.2 Agonism Assay (Data for Each Well of a 384-Well Plate):

The substance solutions (0.75 μl) introduced into an assay plate and 30% DMSO are dissolved in 16 μl of a KRSB+IBMX stimulation solution (1× Krebs-Ringer Bicarbonate Buffer; Sigma-Aldrich # K-4002; including 750 μM 3-isobutyl-1-methylxanthine Sigma-Aldrich # I-7018), and then 15 μl thereof are transferred into a media-free cell culture plate which has been washed with KRSB shortly beforehand.

After incubation at room temperature (RT; volume: ˜15 μl) for 60 minutes, the reaction is then stopped by adding 5 μl of lysis buffer and incubated at RT for a further 20 min (volume: ˜20 μl). The cell lysate is then transferred into a measuring plate and measured in accordance with the manufacturer's information (cyclic AMP kit Cisbio International #62AMPPEC).

### 2. The EP₂ Subtype of the PGE2 Receptor and the Preovulatory Cumulus Expansion

### 2.1. Background:

In the preovulatory antral follicle, the oocyte is surrounded by cumulus cells which form a dense ring of cells around the oocyte. After the LH peak (lutenizing hormone), a series of processes is activated and leads to a large morphological change in this ring of cells composed of cumulus cells. In this case, the cumulus cells form an extracellular matrix which leads to so-called cumulus expansion (Vanderhyden et al. Dev Biol. 1990 August; 140 (2):307-317). This cumulus expansion is an important component of the ovulatory process and of the subsequent possibility of fertilization.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. 1999 Aug. 31; 96 (18):10501-6) show a markedly reduced cumulus expansion and severe subfertility, demonstrating the importance of the prostanoid EP₂ receptor for this process.

### 2.2 Cumulus Expansion Assay In Vitro

Folliculogenesis is induced in immature female mice (strain: CD1 (ICR) from Charles River) at an age of 14-18 days by a single dose (intraperitoneal) of 10 I.U. of PMSG (Pregnant Mare Serum Gonadotropine; Sigma G-4877, Lot 68H0909). 47-50 hours after the injection, the ovaries are removed and the cumulus-oocyte complexes are removed. The cumulus complex is not yet expanded at this stage.

The cumulus-oocyte complexes are then incubated with prostaglandin E2 (PGE2) (0.3 μM), vehicle control (ethanol) or test substances for 20-24 hours. Medium: alpha-MEM medium with 0.1 mM IBMX, pyruvates (0.23 mM) glutamines (2 mM), pen/strep 100 IU/ml pen. and 100 μg/ml strep.) and HSA (8 mg/ml)). Cumulus expansion is then established through the division into four stages (according to Vanderhyden et al. Dev Biol. 1990 August; 140 (2):307-317).

TABLE 1
Example of the biological activity of the compounds of the invention
(measured by the cAMP antagonism assay):

| Substance of | |
|---|---|
| Example | Antagonism [IC₅₀, μM] |
| 1 | 1.1 |
| 5 | 2.7 |
| 6 | 1.3 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 07 090 119.4, filed Jun. 13, 2007, and U.S. Provisional Application Ser. No. 60/943,649, filed Jun. 13, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound of the formula (I) 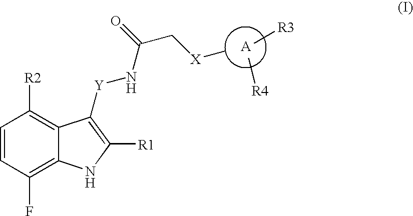
where
A is a C₆-C₁₂ aryl or C₅-C₁₆ heteroaryl radical which may optionally be substituted one or more times by R⁴ and/or R³,
R¹ is a hydrogen, a C₁-C₆-alkyl radical which may optionally be substituted,
R² is a hydrogen, halogen, cyano, —S(O)_{q}—CH₃, where q is 0-2, a C₁-C₄-alkoxy radical or C₁-C₆-alkyl radical, where this radical may be substituted in any way,
R³ is a hydrogen, halogen, amino, an —S(O)ₚ—C₁-C₆-alkyl group, where p is 0-2,
an SO₂NH₂, COOH, CO—NH₂, NH—CO—NH₂, C₁-C₆-acyl, —O—CO—NH(C₁-C₆-alkyl), —O—CO—N(C₁-C₆-alkyl)₂ or NH—CO—C₁-C₆-alkyl radical,
a C₁-C₆-alkyl which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂,
a C₅-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, CO—NH(C₁-C₆-alkyl), CO—N(C₁-C₆-alkyl)₂ or C₁-C₆-alkoxy,
R⁴ is a hydrogen, halogen, amino, —S(O)ₚ—C₁-C₆-alkyl, where p is 0-2,
an SO₂NH₂, COOH, CO—NH₂, NH—CO—NH₂, C₁-C₆-acyl, NH—CO—C₁-C₆-alkyl, —O—CO—NH(C₁-C₆-alkyl), —O—CO—N(C₁-C₆-alkyl)₂,
a C₁-C₆-alkyl which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂,
a C₅-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂,
a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, CO—NH(C₁-C₆-alkyl), CO—N(C₁-C₆-alkyl)₂ or C₁-C₆-alkoxy,
R³ and R⁴ are either in ortho position, meta position or meta, para position relative to one another and together have the meaning —O—CO—S—, —S—CO—O—, CH₂—CO—O—, O—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen, sulfur or an —NH group, and
Y is a —(CH₂)ₙ—, where n is 1-3,
and the isomers, diastereomers, enantiomers and salts thereof, and cyclodextrin clathrates.

2. A compound as claimed in claim 1,
where
A is a C₆-C₁₂ aryl or C₅-C₁₆ heteroaryl radical which may optionally be substituted one or more times by R⁴ and/or R³,
R¹ is a hydrogen or C₁-C₆-alkyl radical which may be substituted one or more times by halogen,
R² is a hydrogen, halogen, cyano, —S(O)_{q}—CH₃, where q is 0-2, a C₁-C₄-alkoxy radical or C₁-C₆-alkyl radical which may be substituted one or more times by halogen,
R³ is a hydrogen, halogen, amino, —S(O)ₚ—C₁-C₆-alkyl, where p is 0-2,
an SO₂NH₂, SO₂NH—CH₃, COOH, CO—NH₂, NH—CO—NH₂, C₁-C₆-acyl, NH—CO—C₁-C₆-alkyl, —O—CO—NH(C₁-C₆-alkyl), —O—CO—N(C₁-C₆-alkyl)₂,
a C₁-C₆-alkyl which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂,
a C₅-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, CO—NH(C₁-C₆-alkyl), CO—N(C₁-C₆-alkyl)₂ or C₁-C₆-alkoxy,
R⁴ is a hydrogen, halogen, amino, —S(O)ₚ—C₁-C₆-alkyl, where p is 0-2,
an SO₂NH₂, COOH, CO—NH₂, NH—CO—NH₂, C₁-C₆-acyl, NH—CO—C₁-C₆-alkyl, —O—CO—NH(C₁-C₆-alkyl), —O—CO—N(C₁-C₆alkyl)₂,
a C₁-C₆-alkyl which may optionally be substituted one or more times, identically or differently, by C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂,
a C₅-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, CO—NH(C₁-C₆-alkyl), CO—N(C₁-C₆-alkyl)₂ or C₁-C₆-alkoxy,
R³ and R⁴ are either in ortho position, meta position or meta, para position relative to one another and together have the meaning —O—CO—S—, —S—CO—O—, CH₂—CO—O—, O—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen, sulfur or an —NH group, and
Y is a —(CH₂)ₙ—, where n is 1-3,
and the isomers, diastereomers, enantiomers and salts thereof, and cyclodextrin clathrates.

3. A compound as claimed in claim 1,
where
A is a C₆-C₁₂ aryl or C₅-C₁₆ heteroaryl radical which may optionally be substituted one or more times by R⁴ and/or R³,
R¹ is a hydrogen or a C₁-C₆-alkyl radical which is substituted one or more times by halogen,
R² is a hydrogen, halogen, cyano, —S(O)_{q}—CH₃, where q is 0-2, a C₁-C₄-alkoxy radical or a C₁-C₆-alkyl group which is substituted one or more times by halogen,
R³ is a hydrogen, halogen, amino, —S(O)ₚ—CH₃, where p is 0-2,
an —S—CF₃—, SO₂NH₂, COOH, CO—NH₂, NH—CO—NH₂, C₁-C₆-acyl, NH—CO—C₁-C₆-alkyl, —O—CO—NH(C₁-C₆-alkyl), —O—CO—N(C₁-C₆-alkyl)₂,
a C₁-C₆-alkyl which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
a C₆-C₁₂-aryl which may optionally be substituted one or more times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
a C₅-C₁₂-heteroaryl which may optionally be substituted one or more times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂, or
a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R⁴ is a hydrogen, halogen, amino, —S(O)ₚ—C₁-C₆-alkyl, where p=0-2, an SO₂NH₂, COOH, CO—NH₂, NH—CO—NH₂, C₁-C₆-acyl-, NH—CO—C₁-C₆-alkyl-, —O—CO—NH(C₁-C₆-alkyl), —O—CO—N(C₁-C₆-alkyl)₂,
a C₁-C₆-alkyl which is optionally substituted one or more times, identically or differently, by C₁-C₆-acyl, C₁-C₆-alkoxy, hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
a C₆-C₁₂-aryl which may optionally be substituted one or more times, identically or differently, by halogen, by C₁-C₆-alkyl, C₃-C₆-cycloalkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂,
a C₅-C₁₂-heteroaryl which may optionally be substituted one or more times, identically or differently, by halogen, by C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH(C₁-C₆-alkyl) or CO—N(C₁-C₆-alkyl)₂, or
a C₃-C₆-cycloalkyl which may optionally be substituted one or more times, identically or differently, by halogen, by C₁-C₆-alkyl, hydroxy, cyano, CO₂—(C₁-C₆-alkyl), C₁-C₆-acyl, N—(C₁-C₆-alkyl)₂, CO—NH(C₁-C₆-alkyl), CO—N(C₁-C₆-alkyl)₂ or C₁-C₆-alkoxy,
R³ and R⁴ are either in ortho position, meta position or meta, para position relative to one another and together have the meaning —O—CO—S—, —S—CO—O—, CH₂—CO—O—, O—CO—CH₂—, —CH₂—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen, sulfur or an —NH group, and
Y is a —(CH₂)ₙ—, where n is 1-3,
and the isomers, diastereomers, enantiomers and salts thereof, and cyclodextrin clathrates.

4. A compound as claimed in claim 1,
where
A is a C₆-C₁₂ aryl or C₅-C₁₆ heteroaryl radical which may optionally be substituted one or more times by R⁴ and/or R³,
R¹ is a hydrogen or a C₁-C₆-alkyl group which is substituted one or more times by halogen,
R² is hydrogen, halogen, cyano, —S(O)_{q}—CH₃, where q is 0-2, a C₁-C₄ alkoxy radical or C₁-C₆-alkyl group which is substituted one or more times by halogen,
R³ is a hydrogen, halogen, amino, —S(O)ₚ—CH₃, where p is 0-2,
an —S—CF₃, SO₂NH₂, COOH, CO—NH₂, NH—CO—NH₂, C₁-C₆-acyl, NH—CO—C₁-C₆-alkyl, —O—CO—NHCH₃, —O—CO—N(CH₃)₂,
a C₁-C₆-alkyl which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
a C₆-C₁₂-aryl which may optionally be substituted one or more times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
a C₅-C₁₂-heteroaryl which may optionally be substituted one or more times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂ or
a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
an —S—CF₃—, SO₂NH₂, COOH, CO—NH₂, NH—CO—NH₂, C₁-C₆-acyl-, NH—CO—C₁-C₆-alkyl, —O—CO—NHCH₃, —O—CO—N(CH₃)₂,
a C₁-C₆-alkyl which may optionally be substituted one or more times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
a C₆-C₁₂-aryl which may optionally be substituted one or more times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
a C₅-C₁₂-heteroaryl which may optionally be substituted one or more times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂, or
a C₃-C₆-cycloalkyl which may optionally be substituted one or more times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R³ and R⁴ are either in ortho position, meta position or meta, para position relative to one another and together have the meaning —O—CO—S—, —S—CO—O—, CH₂—CO—O—, O—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen, sulfur or an —NH group, and
Y is a —(CH₂)ₙ—, where n is 1-3,
and the isomers, diastereomers, enantiomers and salts thereof, and cyclodextrin clathrates.

5. A compound as claimed in claim 1,
where
A is a phenyl, naphthyl or C₅-C₁₆ heteroaryl radical which may be substituted by R³ and/or R⁴,
R¹ is a hydrogen or a C₁-C₆-alkyl group which may be substituted one or more times by halogen,
R² is a hydrogen, halogen, cyano, —S(O)_{q}—CH₃, where q is 0-2, a C₁-C₄-alkoxy radical or C₁-C₆-alkyl group,
R³ is a hydrogen, halogen, amino, —S(O)ₚ—CH₃, where p is 0-2,
an —S—CF₃—, SO₂NH₂, COOH, CO—NH₂, NH—CO—NH₂, C₁-C₆-acyl, NH—CO—C₁-C₆-alkyl, —O—CO—NHCH₃, —O—CO—N(CH₃)₂,
a C₁-C₆-alkyl which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
a C₆-C₁₂-aryl which may optionally be substituted once, twice, three, four or five times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
a C₅-C₁₂-heteroaryl which may optionally be substituted once, twice, three, four or five times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂ or
a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
an —S—CF₃—, SO₂NH₂, COOH, CO—NH₂, NH—CO—NH₂, C₁-C₆-acyl-, NH—CO—C₁-C₆-alkyl-, —O—CO—NHCH₃, —O—CO—N(CH₃)₂,
a C₁-C₆-alkyl which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
a C₁-C₆-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₆-alkyl), O—CO—(C₁-C₆-alkyl), N—(C₁-C₆-alkyl)₂, NH—(C₁-C₆-alkyl), CO—NH(C₅-C₁₂-heteroaryl), CO—NH(C₁-C₆-alkyl), CO—N(C₁-C₆-alkyl)₂,
a C₆-C₁₂-aryl which may optionally be substituted once, twice, three, four or five times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
a C₅-C₁₂-heteroaryl which may optionally be substituted once, twice, three, four or five times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂, or
a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R³ and R⁴ are either in ortho position, meta position or meta, para position relative to one another and together have the meaning —O—CO—S—, —S—CO—O—, CH₂—CO—O—, O—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen, sulfur or an —NH group, and
Y is a —(CH₂)ₙ—, where n is 1-3,
and the isomers, diastereomers, enantiomers and salts thereof, and cyclodextrin clathrates.

6. A compound as claimed in claim 1,
where
A is a phenyl, naphthyl or C₅-C₁₆ heteroaryl radical which is substituted by R³ and/or R⁴,
R¹ is a hydrogen or a C₁-C₆-alkyl radical which is substituted one or more times by halogen,
R² is a hydrogen, halogen, cyano, —S(O)_{q}—CH₃, where q is 0-2, a C₁-C₄ alkoxy radical or C₁-C₆-alkyl radical which is substituted one or more times by halogen,
R³ is a hydrogen, halogen, amino, —S(O)ₚ—CH₃, where p is 0-2,
an —S—CF₃—, SO₂NH₂, COOH, CO—NH₂, NH—CO—NH₂, C₁-C₆-acyl, NH—CO—C₁-C₆-alkyl, —O—CO—NHCH₃, —O—CO—N(CH₃)₂,
a C₁-C₆-alkyl which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
a C₁-C₄-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₄-alkyl), O—CO—(C₁-C₄-alkyl), N—(C₁-C₄-alkyl)₂, NH—(C₁-C₄-alkyl), CO—NH(C₅-C₁₂-heteroaryl), CO—NH(C₁-C₄-alkyl), CO—N(C₁-C₄-alkyl)₂,
a C₆-C₁₂-aryl which may optionally be substituted once, twice, three, four or five times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
a C₅-C₁₂-heteroaryl which may optionally be substituted once, twice, three, four or five times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂ or
a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
an —S—CF₃—, SO₂NH₂, COOH, CO—NH₂, NH—CO—NH₂, C₁-C₆-acyl, NH—CO—C₁-C₆-alkyl, —O—CO—NHCH₃, —O—CO—N(CH₃)₂,
a C₁-C₆-alkyl which may optionally be substituted once, twice, three, four or five times, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
a C₁-C₄-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₄-alkyl), O—CO—(C₁-C₄-alkyl), N—(C₁-C₄-alkyl)₂, NH—(C₁-C₄-alkyl), CO—NH(C₅-C₁₂-heteroaryl), CO—NH(C₁-C₄-alkyl), CO—N(C₁-C₄-alkyl)₂,
a C₆-C₁₂-aryl which may optionally be substituted once, twice, three, four or five times, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
a C₅-C₁₂-heteroaryl which may optionally be substituted once, twice, three, four or five times, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂, or
a C₃-C₆-cycloalkyl which may optionally be substituted once, twice, three, four or five times, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R³ and R⁴ are either in ortho position, meta position or meta, para position relative to one another and together have the meaning —O—CO—S—, —S—CO—O—, CH₂—CO—O—, O—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen, sulfur or an —NH group, and
Y is a —(CH₂)ₙ—, where n is 1-3,
and the isomers, diastereomers, enantiomers and salts thereof, and cyclodextrin clathrates.

7. A compound as claimed in claim 1,
where
A is a phenyl, naphthyl or C₅-C₁₆ heteroaryl radical which is substituted by R³ and/or R⁴,
R¹ is a hydrogen or a C₁-C₆-alkyl radical which is substituted one or more times by halogen,
R² is hydrogen, halogen, cyano, —S(O)_{q}—CH₃, where q is 0-2, a C₁-C₄-alkoxy radical or C₁-C₆-alkyl radical which is substituted one, or more times by halogen,
R³ is a hydrogen, halogen, amino, —S(O)ₚ—CH₃, where p is 0-2,
an —S—CF₃—, SO₂NH₂, COOH, CO—NH₂, NH—CO—NH₂, C₁-C₆-acyl, NH—CO—C₁-C₆-alkyl, —O—CO—NHCH₃, —O—CO—N(CH₃)₂,
a C₁-C₆-alkyl which may optionally be substituted once or twice, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
a C₁-C₄-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₄-alkyl), O—CO—(C₁-C₄-alkyl), N—(C₁-C₄-alkyl)₂, NH—(C₁-C₄-alkyl), CO—NH(C₅-C₁₂-heteroaryl), CO—NH(C₁-C₄-alkyl), CO—N(C₁-C₄-alkyl)₂,
a C₆-C₁₂-aryl which may optionally be substituted once or twice, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
a C₅-C₁₂-heteroaryl which may optionally be substituted once or twice, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂ or
a C₃-C₆-cycloalkyl which may optionally be substituted once or twice, identically or differently, by halogen, by C₁-C₄-alkyl, hydroxy, cyano, CO₂—(C₁-C₄-alkyl), C₁-C₄-alkyl, N—(C₁-C₄-alkyl)₂, CO—NH(C₁-C₄-alkyl), CO—N(C₁-C₄-alkyl)₂ or C₁-C₄-alkoxy,
R⁴ is a hydrogen, halogen, amino, —S(O)ₚ—CH₃, where p is 0-2,
an —S—CF₃—, SO₂NH₂, COOH, CO—NH₂, NH—CO—NH₂, C₁-C₆-acyl-, NH—CO—C₁-C₆-alkyl-, —O—CO—NHCH₃, —O—CO—N(CH₃)₂,
a C₁-C₆-alkyl which may optionally be substituted once or twice, identically or differently, by C₁-C₄-acyl, C₁-C₄-alkoxy, hydroxy, cyano, CO₂—(C₁-C₄-alkyl), N—(C₁-C₄-alkyl)₂, COOH, CO—NH₂, CO—NH(C₁-C₄-alkyl) or by CO—N(C₁-C₄-alkyl)₂,
a C₁-C₄-alkoxy which may optionally be substituted one or more times, identically or differently, by hydroxy, cyano, CO₂—(C₁-C₆-alkyl), N—(C₁-C₆-alkyl)₂, COOH, CO—NH₂, CO—NH(C₁-C₆-alkyl) or by CO—N(C₁-C₆-alkyl)₂,
an —O—C₆-C₁₂-aryl, —CH₂—O—C₆-C₁₂-aryl, —O—C₅-C₁₆-heteroaryl, hydroxy, cyano, CO₂—(C₁-C₄-alkyl), O—CO—(C₁-C₄-alkyl), N—(C₁-C₄-alkyl)₂, NH—(C₁-C₄-alkyl), CO—NH(C₅-C₁₂-heteroaryl), CO—NH(C₁-C₄-alkyl), CO—N(C₁-C₄-alkyl)₂,
a C₆-C₁₂-aryl which may optionally be substituted once or twice, identically or differently, by halogen, by C₁-C₄-alkyl, C₃-C₆-cycloalkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂,
a C₅-C₁₂-heteroaryl which may optionally be substituted once or twice, identically or differently, by halogen, by C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH(C₁-C₄-alkyl) or CO—N(C₁-C₄-alkyl)₂, or
a C₃-C₆-cycloalkyl which may optionally be substituted once or twice, identically or differently, by halogen, by C₁-C₄-alkyl, hydroxy, cyano, CO₂—(C₁-C₄-alkyl), C₁-C₄-acyl, N—(C₁-C₄-alkyl)₂, CO—NH(C₁-C₄-alkyl), CO—N(C₁-C₄-alkyl)₂ or C₁-C₄-alkoxy,
R³ and R⁴ are either in ortho position, meta position or meta, para position relative to one another and together have the meaning —O—CO—S—, —S—CO—O—, CH₂—CO—O—, O—CO—CH₂—, O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
X is an oxygen or sulfur, and
Y is a —(CH₂)ₙ— group, where n is 1-3,
and the isomers, diastereomers, enantiomers and salts thereof, and cyclodextrin clathrates.

8. A compound as claimed in claim 1 selected from a group which comprises the following compounds:
1. N-[2-(7-fluoro-2,4-dimethyl-1H-indol-3-yl)ethyl]-2-(4-methoxyphenoxy)-acetamide
2. N-[2-(7-fluoro-2,4-dimethyl-1H-indol-3-yl)ethyl]-2-(3-methoxyphenoxy)-acetamide
3. N-[2-(7-fluoro-2,4-dimethyl-1H-indol-3-yl)ethyl]-2-(2-methoxyphenoxy)-acetamide
4. N-[2-(7-fluoro-2,4-dimethyl-1H-indol-3-yl)ethyl]-2-(4-fluorophenylthio)acetamide
5. N-[2-(7-fluoro-2,4-dimethyl-1H-indol-3-yl)ethyl]-2-phenoxyacetamide
6. 2-(4-chlorophenoxy)-N-[2-(7-fluoro-2,4-dimethyl-1H-indol-3-yl)ethyl]-acetamide
7. 2-(2-chlorophenoxy)-N-[2-(7-fluoro-2,4-dimethyl-1H-indol-3-yl)ethyl]-acetamide
8. N-[2-(7-fluoro-2,4-dimethyl-1H-indol-3-yl)ethyl]-2-(isoquinolin-7-yloxy)-acetamide
9. 2-(3-acetylaminophenoxy)-N-[2-(7-fluoro-2,4-dimethyl-1H-indol-3-yl)ethyl]-acetamide
10. N-[2-(7-fluoro-2,4-dimethyl-1H-indol-3-yl)ethyl]-2-(5,6,7,8-tetrahydro-naphthalen-2-yloxy)acetamide
11. 4-{[2-(7-fluoro-2,4-dimethyl-1H-indol-3-yl)ethylcarbamoyl]methoxy}benzoic acid methyl ester
12. 2-(biphenyl-4-yloxy)-N-[2-(7-fluoro-2,4-dimethyl-1H-indol-3-yl)ethyl]-acetamide
13. 2-(4-acetylaminophenoxy)-N-[2-(7-fluoro-2,4-dimethyl-1H-indol-3-yl)ethyl]-acetamide
14. N-[2-(7-fluoro-2,4-dimethyl-1H-indol-3-yl)ethyl]-2-(2-oxo-benzo[1,3]oxathiol-6-yloxy)acetamide
15. 2-(3-cyanophenoxy)-N-[2-(7-fluoro-2,4-dimethyl-1H-indol-3-yl)ethyl]-acetamide
16. N-[2-(7-fluoro-2,4-dimethyl-1H-indol-3-yl)ethyl]-2-(quinolin-8-yloxy)-acetamide
17. 2-(5-chloro-2-methylphenoxy)-N-[2-(7-fluoro-2,4-dimethyl-1H-indol-3-yl)-ethyl]acetamide
18. N-[2-(7-fluoro-2,4-dimethyl-1H-indol-3-yl)ethyl]-2-(1-oxo-indan-5-yloxy)-acetamide
19. N-[2-(7-fluoro-2,4-dimethyl-1H-indol-3-yl)ethyl]-2-o-tolyloxyacetamide
20. 2-(4-chloro-2-methylphenoxy)-N-[2-(7-fluoro-2,4-dimethyl-1H-indol-3-yl)-ethyl]acetamide
21. N-[2-(7-fluoro-2,4-dimethyl-1H-indol-3-yl)ethyl]-2-m-tolyloxyacetamide
22. N-[2-(7-fluoro-2,4-dimethyl-1H-indol-3-yl)ethyl]-2-p-tolyloxyacetamide
23. N-[2-(7-fluoro-2,4-dimethyl-1H-indol-3-yl)ethyl]-2-(naphthalen-2-yloxy)-acetamide
24. N-[2-(7-fluoro-2,4-dimethyl-1H-indol-3-yl)ethyl]-2-(5,6,7,8-tetrahydro-naphthalen-1-yloxy)acetamide
25. N-[2-(7-fluoro-2,4-dimethyl-1H-indol-3-yl)ethyl]-2-(naphthalen-1-yloxy)-acetamide
26. 2-(4-chloro-3-methylphenoxy)-N-[2-(7-fluoro-2,4-dimethyl-1H-indol-3-yl)-ethyl]acetamide
27. N-[2-(7-fluoro-2,4-dimethyl-1H-indol-3-yl)ethyl]-2-(3-fluorophenoxy)-acetamide
28. 2-(3,5-dimethoxyphenoxy)-N-[2-(7-fluoro-2,4-dimethyl-1H-indol-3-yl)ethyl]-acetamide
29. N-[2-(7-fluoro-2,4-dimethyl-1H-indol-3-yl)ethyl]-2-(4-imidazol-1-ylphenoxy)-acetamide
30. 2-(3,4-dimethoxyphenoxy)-N-[2-(7-fluoro-2,4-dimethyl-1H-indol-3-yl)ethyl]-acetamide
31. 2-(biphenyl-2-yloxy)-N-[2-(7-fluoro-2,4-dimethyl-1H-indol-3-yl)ethyl]-acetamide
32. N-[2-(7-fluoro-2,4-dimethyl-1H-indol-3-yl)ethyl]-2-(1H-indol-4-yloxy)-acetamide
33. N-[2-(7-fluoro-2,4-dimethyl-1H-indol-3-yl)ethyl]-2-(1H-indol-5-yloxy)-acetamide
34. 2-(4-tert-butylphenoxy)-N-[2-(7-fluoro-2,4-dimethyl-1H-indol-3-yl)ethyl]-acetamide
35. 2-(4-{[2-(7-fluoro-2,4-dimethyl-1H-indol-3-yl)ethylcarbamoyl]methoxy}-phenyl)acetamide
36. 2-(benzo[1,3]dioxol-5-yloxy)-N-[2-(7-fluoro-2,4-dimethyl-1H-indol-3-yl)ethyl]-acetamide
37. N-[2-(7-fluoro-2,4-dimethyl-1H-indol-3-yl)ethyl]-2-(2-pyrrol-1-ylphenoxy)-acetamide
38. N-[2-(7-fluoro-2,4-dimethyl-1H-indol-3-yl)ethyl]-2-(4-isopropylphenoxy)-acetamide
39. N-[2-(7-fluor-2,4-dimethyl-1H-indol-3-yl)ethyl]-2-(quinazolin-4-yloxy)-acetamide
40. N-[2-(7-fluoro-2,4-dimethyl-1H-indol-3-yl)ethyl]-2-(6-methylpyridin-3-yloxy)-acetamide
41. N-[2-(7-fluoro-2,4-dimethyl-1H-indol-3-yl)ethyl]-2-(3-piperidin-4-ylphenoxy)-acetamide
42. N-[2-(7-fluoro-2,4-dimethyl-1H-indol-3-yl)ethyl]-2-(indan-5-yloxy)acetamide
43. N-[2-(7-fluoro-2,4-dimethyl-1H-indol-3-yl)ethyl]-2-(2-oxo-2,3-dihydro-benzofuran-5-yloxy)acetamide
44. 2-(3,4-difluorophenoxy)-N-[2-(7-fluoro-2,4-dimethyl-1H-indol-3-yl)ethyl]-acetamide
45. 2-(2-dimethylaminomethylphenoxy)-N-[2-(7-fluoro-2,4-dimethyl-1H-indol-3-yl)ethyl]acetamide
46. 2-(3-dimethylaminophenoxy)-N-[2-(7-fluoro-2,4-dimethyl-1H-indol-3-yl)ethyl]-acetamide
47. 2-(4-dimethylaminomethylphenoxy)-N-[2-(7-fluoro-2,4-dimethyl-1H-indol-3-yl)ethyl]acetamide
48. 2-(2,3-dimethoxyphenoxy)-N-[2-(7-fluoro-2,4-dimethyl-1H-indol-3-yl)ethyl]-acetamide
49. N-[2-(7-fluoro-2,4-dimethyl-1H-indol-3-yl)ethyl]-2-[2-(2H-pyrazol-3-yl)-phenoxy]acetamide
50. N-[2-(7-fluoro-2,4-dimethyl-1H-indol-3-yl)ethyl]-2-(4-pyrrol-1-ylphenoxy)-acetamide
51. N-[2-(7-fluoro-2,4-dimethyl-1H-indol-3-yl)ethyl]-2-(2-isoxazol-5-ylphenoxy)-acetamide
52. N-[2-(7-fluoro-2,4-dimethyl-1H-indol-3-yl)ethyl]-2-(2-methylbenzothiazol-5-yloxy)acetamide
53. 2-(2-chloro-4-fluorophenoxy)-N-[2-(7-fluoro-2,4-dimethyl-1H-indol-3-yl)ethyl]-acetamide
54. N-[2-(7-fluoro-2,4-dimethyl-1H-indol-3-yl)ethyl]-2-(3-morpholin-4-ylphenoxy)-acetamide
55. 2-(3,4-dimethylphenoxy)-N-[2-(7-fluoro-2,4-dimethyl-1H-indol-3-yl)ethyl]-acetamide
56. N-[2-(7-fluoro-2,4-dimethyl-1H-indol-3-yl)ethyl]-2-(4-methylpyridin-2-yloxy)-acetamide
57. N-[2-(7-fluoro-2,4-dimethyl-1H-indol-3-yl)ethyl]-2-(isoquinolin-5-yloxy)-acetamide
58. N-[2-(7-fluoro-2,4-dimethyl-1H-indol-3-yl)ethyl]-2-(4-phenoxyphenoxy)-acetamide
59. N-[2-(7-fluoro-2,4-dimethyl-1H-indol-3-yl)ethyl]-2-(quinolin-4-yloxy)acetamide
60. N-[2-(7-fluoro-2,4-dimethyl-1H-indol-3-yl)ethyl]-2-(4-oxazol-2-ylphenoxy)-acetamide
61. 2-(4-cyanophenoxy)-N-[2-(7-fluoro-2,4-dimethyl-1H-indol-3-yl)ethyl]-acetamide
62. 2-(2-chloro-5-methylphenoxy)-N-[2-(7-fluoro-2,4-dimethyl-1H-indol-3-yl)-ethyl]acetamide
63. 2-(biphenyl-3-yloxy)-N-[2-(7-fluoro-2,4-dimethyl-1H-indol-3-yl)ethyl]-acetamide
64. N-[2-(7-fluoro-2,4-dimethyl-1H-indol-3-yl)ethyl]-2-(4-fluorophenoxy)-acetamide
65. 2-(3-chlorophenoxy)-N-[2-(7-fluoro-2,4-dimethyl-1H-indol-3-yl)ethyl]-acetamide
66. 2-(3-chloro-4-fluorophenoxy)-N-[2-(7-fluoro-2,4-dimethyl-1H-indol-3-yl)ethyl]-acetamide
67. N-[2-(7-fluoro-2,4-dimethyl-1H-indol-3-yl)ethyl]-2-(3-methoxy-5-methyl-phenoxy)acetamide
68. N-[2-(7-fluoro-2,4-dimethyl-1H-indol-3-yl)ethyl]-2-(quinolin-7-yloxy)-acetamide
69. N-[2-(7-fluoro-2,4-dimethyl-1H-indol-3-yl)ethyl]-2-(quinolin-6-yloxy)-acetamide
70. N-[2-(7-fluoro-2,4-dimethyl-1H-indol-3-yl)ethyl]-2-(4-[1,2,4]triazol-1-yl-phenoxy)acetamide
71. N-[2-(7-fluoro-2,4-dimethyl-1H-indol-3-yl)ethyl]-2-(3-piperazin-1-ylphenoxy)-acetamide
72. N-[2-(7-fluoro-2,4-dimethyl-1H-indol-3-yl)ethyl]-2-(3-piperazin-1-ylphenoxy)-acetamide
73. N-[2-(7-fluoro-2,4-dimethyl-1H-indol-3-yl)ethyl]-2-(4-fluoro-2-methyl-1H-indol-5-yloxy)acetamide
74. N-[2-(7-fluoro-2,4-dimethyl-1H-indol-3-yl)ethyl]-2-(4-piperazin-1-ylphenoxy)-acetamide
75. 2-(3-chloro-2-methyl-phenoxy)-N-[2-(7-fluoro-2,4-dimethyl-1H-indol-3-yl)-ethyl]acetamide
76. 2-(2,3-difluorophenoxy)-N-[2-(7-fluoro-2,4-dimethyl-1H-indol-3-yl)ethyl]-acetamide
77. 2-(3-chloro-2-fluorophenoxy)-N-[2-(7-fluoro-2,4-dimethyl-1H-indol-3-yl)ethyl]-acetamide
78. N-[2-(7-fluoro-2,4-dimethyl-1H-indol-3-yl)ethyl]-2-(3-carbamoylamino-phenoxy)acetamide
79. 2-(9H-carbazol-3-yloxy)-N-[2-(7-fluoro-2,4-dimethyl-1H-indol-3-yl)ethyl]-acetamide
80. 2-(4-bromo-3-methoxyphenoxy)-N-[2-(7-fluoro-2,4-dimethyl-1H-indol-3-yl)-ethyl]acetamide
81. 2-(3-chloro-5-fluorophenoxy)-N-[2-(7-fluoro-2,4-dimethyl-1H-indol-3-yl)ethyl]-acetamide
82. 2-(2-chloro-4-methylphenoxy)-N-[2-(7-fluoro-2,4-dimethyl-1H-indol-3-yl)-ethyl]acetamide
83. 2-(3-chloro-4-methylphenoxy)-N-[2-(7-fluoro-2,4-dimethyl-1H-indol-3-yl)-ethyl]acetamide
84. N-[2-(7-fluoro-2,4-dimethyl-1H-indol-3-yl)ethyl]-2-(4-fluoro-3-methyl-phenoxy)acetamide
85. N-[2-(7-fluoro-2,4-dimethyl-1H-indol-3-yl)ethyl]-2-(3-fluoro-4-methyl-phenoxy)acetamide
86. N-[2-(7-fluoro-2,4-dimethyl-1H-indol-3-yl)ethyl]-2-(4-tetrazol-1-ylphenoxy)-acetamide
87. N-[2-(7-fluoro-2,4-dimethyl-1H-indol-3-yl)ethyl]-2-[(3-dimethylcarbamoyl)oxy]-phenoxy)acetamide
88. N-[2-(7-fluoro-2,4-dimethyl-1H-indol-3-yl)ethyl]-2-(4-fluoro-2-methyl-phenoxy)acetamide
89. N-[2-(7-fluoro-2,4-dimethyl-1H-indol-3-yl)ethyl]-2-(3-fluoro-5-methyl-phenoxy)acetamide
90. N-[2-(7-fluoro-2,4-dimethyl-1H-indol-3-yl)ethyl]-2-(2-oxo-2,3-dihydro-benzooxazol-6-yloxy)acetamide
91. N-[2-(7-fluoro-2,4-dimethyl-1H-indol-3-yl)ethyl]-2-[(4-dimethylcarbamoyl)oxy]-phenoxy)acetamide
92. 4-{[2-(7-fluoro-2,4-dimethyl-1H-indol-3-yl)ethylcarbamoyl]methoxy}-benzamide
93. 2-(9H-carbazol-2-yloxy)-N-[2-(7-fluoro-2,4-dimethyl-1H-indol-3-yl)ethyl]-acetamide

9. (canceled)

10. (canceled)

11. The use of the medicaments as set forth in claim 22, wherein the medicament is used for the treatment and prophylaxis of disorders.

12. A method for the treatment or prophylaxis of a disorder connected with the EP₂ receptor comprising administering to a subject in need thereof an effective amount of a compound of claim 1.

13. A method for the treatment or prophylaxis of a fertility impairment comprising administering to a subject in need thereof an effective amount of a compound of claim 1.

14. A method for the treatment or prophylaxis of painful menstruation comprising administering to a subject in need thereof an effective amount of a compound of claim 1.

15. A method for the treatment or prophylaxis of endometriosis comprising administering to a subject in need thereof an effective amount of a compound of claim 1.

16. A method for modulating the EP₂ receptor comprising administering to a subject in need thereof an effective amount of a compound of claim 1.

17. A method for the treatment or prophylaxis of pain comprising administering to a subject in need thereof an effective amount of a compound of claim 1.

18. A method for controlling fertility/contraception comprising administering to a subject in need thereof an effective amount of a compound of claim 1.

19. A method for the treatment or prophylaxis of osteoporosis comprising administering to a subject in need thereof an effective amount of a compound of claim 1.

20. A method for the treatment or prophylaxis of cancer comprising administering to a subject in need thereof an effective amount of a compound of claim 1.

21. The use of the compounds of the general formula I as claimed in claim 1 in the form of a pharmaceutical product for enteral, parenteral, vaginal and oral administration.

22. A pharmaceutical composition comprising a compound of claim 1 and a pharmaceutically acceptable carrier.
